(19) United States
(12) Patent Application Publication
(10) Pub. No.: US 2022/0205003 A1
(43) Pub. Date: Jun. 30, 2022

(54) MICROORGANISM WITH ENHANCED L-HISTIDINE PRODUCTION CAPACITY AND METHOD FOR PRODUCING HISTIDINE BY USING SAME

(71) Applicant: CJ CHEILJEDANG CORPORATION, Seoul (KR)

(72) Inventors: Lan Huh, Seoul (KR); Nara Kwon, Seoul (KR); Chang Il Seo, Seoul (KR)

(73) Assignee: CJ CHEILJEDANG CORPORATION, Seoul (KR)

(21) Appl. No.: 17/605,648

(22) PCT Filed: Mar. 10, 2020
(86) PCT No.: PCT/KR2020/003317

§ 371 Date: Oct. 22, 2021

(30) Foreign Application Priority Data
Apr. 22, 2019 (KR) 10-2019-0046934

(51) Int. Cl. C12P 13/24; C07K 14/34; C12N 1/20; C12N 15/52; C12N 15/77; C12N 9/10; C12N 9/06

(52) U.S. Cl. CPC C12P 13/24; C12R 2001/15; C12N 1/205; C12N 15/52; C12N 15/77; C12N 9/1014; C12N 9/0016; C12N 9/1029; C12N 9/1077; C12N 9/13; C12Y 104/04002; C12Y 201/0201; C12Y 203/01181; C12Y 208/01008; C07K 14/34

(57) ABSTRACT

Provided are a microorganism having an enhanced L-histidine producing ability and a method of producing histidine using the same.

### BACKGROUND OF THE INVENTION

### 1. Field of the Invention

The present disclosure relates to a microorganism having an enhanced L-histidine producing ability and a method of producing histidine using the same.

### 2. Description of the Related Art

L-Histidine is one of the 20 standard amino acids, the majority of which, from a nutritional point of view, are not required for adults, but it is classified as an essential amino acid for growing children. Additionally, L-histidine is involved in important physiological processes such as antioxidation, immune regulation, etc., and thus is used in the medical industry, such as an agent for treating gastric ulcer, a raw material for a cardiovascular agent, amino acid sap, etc.

Histidine is mainly found in hemoglobin, and is primarily produced by a protein hydrolysis extraction method using blood meal as a raw material. However, it has disadvantages such as low efficiency, environmental pollution, etc. On the other hand, L-histidine can be produced through microbial fermentation, but large-scale industrialization has not yet been accomplished. This is because the biosynthesis of L-histidine competes with a nucleotide synthesis precursor, i.e., PRPP, and has a complicated biosynthetic process and regulatory mechanism requiring high energy.

The L-histidine producing ability of a microorganism used in a fermentation method has been improved by a mutagenic and mutant selection method and a method of regulating the metabolism of a strain through genetic modification. Recently, the production of histidine using microorganisms has been known to be accomplished by biosynthesis from PRPP via several steps. However, an ATP phosphoribosyl transferase, which is the first enzyme involved in the biosynthesis of histidine, has feedback inhibition by the final product, i.e., histidine, or a derivative thereof, and this is a problem in the industrial mass production of L-histidine (International Patent Publication No. WO 2014-029376). Due to this complicated biosynthetic process and regulatory mechanism, an approach from various perspectives related to microbial metabolism is necessary in order to produce L-histidine through microbial culture.

To develop a microorganism which may utilize glycine by importing the glycine exported out of cells, the present inventors introduced a glycine transporter cycA derived from Corynebacterium ammoniagenes, and as result, they have completed a microorganism producing L-histidine with a high yield.

### SUMMARY OF THE INVENTION

An object of the present disclosure is to provide a microorganism of the genus Corynebacterium for producing L-histidine, the microorganism having an enhanced glycine transporter activity.

Another object of the present disclosure is to provide a composition for producing L-histidine, the composition including the microorganism of the present disclosure.

Still another object of the present disclosure is to provide a method of producing L-histidine, the method including the step of culturing the microorganism of the present disclosure.

Still another object of the present disclosure is to provide use of the microorganism of the genus Corynebacterium having an enhanced glycine transporter activity in the production of L-histidine.

### DETAILED DESCRIPTION OF THE PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Further, those skilled in the art will recognize, or be able to ascertain using no more than routine experimentation, many equivalents to the specific embodiments of the disclosure described herein. Further, these equivalents should be interpreted to fall within the present disclosure.

One aspect of the present disclosure provides a microorganism of the genus Corynebacterium for producing L-histidine, the microorganism having an enhanced glycine transporter activity.

As used herein, the term “glycine transporter” may include any protein capable of introducing glycine into cells, and it may be specifically D-serine/D-alanine/glycine transporter. The glycine transporter may be interchangeably used with D-serine/D-alanine/glycine transporter or a protein for glycine uptake.

The “D-serine/D-alanine/glycine transporter” is a protein that may be involved in the transport of all of serine, alanine, and glycine, and information thereof may be obtained by searching for the D-serine/D-alanine/glycine transporter sequence in a known database such as NCBI Genbank, etc. The transporter may be specifically CycA or AapA, and more specifically a CycA protein, but is not limited thereto.

As used herein, the term “CycA protein” refers to a protein involved in serine, alanine, and glycine uptake. The CycA protein is encoded by a cycA gene, and the cycA gene is known to exist in microorganisms, such as Escherichia coli, Kiebsiella pneumoniae, Mycobacterum bovis, Salmonella enterica, Erwinia amylovora, Corynebacterium ammoniagenes, etc.

With respect to the objects of the present disclosure, the CycA protein of the present disclosure may include any protein as long as it may enhance the histidine producing ability. Specifically, the CycA protein may be derived from a microorganism of the genus Corynebacterium or the genus Escherichia, and more specifically Corynebacterium ammoniagenes, but is not limited thereto. Corynebacterium ammoniagenes is the same species as Brevibacterium ammoniagenes, and has been classified in the same taxon as Corynebacterium stationis and Bevibacterium stationis (International Journal of Systematic and Evolutionary Microbiology 60:874-879). Additionally, Brevibacterium ammoniagenes has been renamed as Corynebacterium stationis.

Accordingly, as used herein, the terms Corynebacterium ammoniagenes, Brevibacterium ammoniagenes, Corynebacterium stationis, and Brevibacterium stationis may be used interchangeably.

The CycA protein of the present disclosure may include an amino acid sequence of SEQ ID NO: 1 or an amino acid sequence having 70% or more homology or identity thereto.

Specifically, the CycA protein may include an amino acid sequence of SEQ ID NO: 1 or an amino acid sequence having at least 70%, 75%, 80%, 85%, 86%, 87%, 88%, 89%, 90%, 91%, 92%, 93%, 94%, 95%, 96%, 97%, 98%, or 99% homology or identity to the amino acid sequence of SEQ ID NO: 1. Additionally, it is apparent that any amino acid sequence in which part of the sequence is deleted, modified, substituted, or added may also fall within the scope of the present disclosure, as long as the amino acid sequence has such a homology or identity and exhibits efficacy corresponding to that of the above protein.

Further, a probe that may be prepared from a known gene sequence, for example, any polypeptide encoded by a polynucleotide which may hybridize with a sequence complementary to all or part of a nucleotide sequence under stringent conditions to encode the polypeptide, may include polypeptides having the serine, alanine, and glycine uptake activity without limitation.

In other words, although it is described as “a protein or polypeptide including an amino acid sequence described by a specific sequence number”, “a protein or polypeptide consisting of an amino acid sequence described by a specific sequence number”, or a “protein or polypeptide having an amino acid sequence described by a specific sequence number” in the present disclosure, it is apparent that any protein having an amino acid sequence in which part of the sequence is deleted, modified, substituted, conservatively substituted, or added may be used in the present disclosure as long as it has the same or corresponding activity as the polypeptide consisting of the amino acid sequence of the corresponding sequence number. For example, it may be a case where the N-terminus and/or C-terminus of the amino acid sequence is added with a sequence that does not alter the function of the protein, a naturally occurring mutation, a silent mutation thereof, or a conservative substitution.

As used herein, the term “conservative substitution” refers to substitution of an amino acid with another amino acid having similar structural and/or chemical properties. Such amino acid substitution may generally occur based on similarity of polarity, charge, solubility, hydrophobicity, hydrophilicity, and/or amphipathic nature of a residue. For example, positively charged (basic) amino acids include arginine, lysine, and histidine; negatively charged (acidic) amino acids include glutamic acid and aspartic acid; aromatic amino acids include phenylalanine, tryptophan, and tyrosine; and hydrophobic amino acids include alanine, valine, isoleucine, leucine, methionine, phenylalanine, tyrosine, and tryptophan.

As used herein, the term “polynucleotide” has a meaning which collectively includes DNA or RNA molecules. Nucleotides, which are the basic structural units of the polynucleotides, include not only natural nucleotides but also modified analogs thereof in which sugar or base sites are modified (see Scheit, Nucleotide Analogs, John Wiley, New York (1980); Uhlman and Peyman, Chemical Reviews, 90:543-584 (1990)).

The polynucleotide may be a polynucleotide encoding the CycA protein of the present disclosure, or may be a polynucleotide encoding a polypeptide having at least 70%, 75%, 80%, 85%, 86%, 87%, 88%, 89%, 90%, 91%, 92%, 93%, 94%, 95%, 96%, 97%, 98%, or 99% homology or identity to the CycA protein of the present disclosure. Specifically, for example, the polynucleotide encoding the protein including the amino acid sequence of SEQ ID NO: 1 or the amino acid sequence having 70% or more homology or identity to SEQ ID NO: 1 may be a polynucleotide including a polynucleotide sequence of SEQ ID NO: 2 or having at least 70%, 75%, 80%, 85%, 86%, 87%, 88%, 89%, 90%, 91%, 92%, 93%, 94%, 95%, 96%, 97%, 98%, or 99% homology or identity to the polynucleotide sequence of SEQ ID NO: 2.

Additionally, based on codon degeneracy, it is apparent that polynucleotides which may be translated into proteins including the amino acid sequence of SEQ ID NO: 1 or an amino acid sequence having 70% or more identity to SEQ ID NO: 1, or proteins having a homology or identity thereto may also be included. Additionally, the polynucleotide of the present disclosure may include a probe that may be prepared from a known gene sequence, for example, any polynucleotide sequence which may hybridize with a sequence complementary to all or part of the polynucleotide sequence under stringent conditions to encode proteins including the amino acid sequence having 70% or more identity to the amino acid sequence of SEQ ID NO: 1, without limitation. The “stringent conditions” refer to conditions under which specific hybridization between polynucleotides is allowed. Such conditions are specifically described in the literature (e.g., J. Sambrook et al., Molecular Cloning, A Laboratory Manual, 2nd Edition, Cold Spring Harbor Laboratory Press, Cold Spring Harbor, N.Y., 1989: F. M. Ausubel et al., Current Protocols in Molecular Biology, John Wiley & Sons, Inc., New York). For example, the stringent conditions may include conditions under which genes having a high homology or identity of 70% or higher, 80% or higher, specifically 85% or higher, specifically 90% or higher, more specifically 95% or higher, much more specifically 97% or higher, and still much more specifically 99% or higher are hybridized with each other, and genes having a homology or identity lower than the above homologies or identities are not hybridized with each other, or washing conditions of Southern hybridization, that is, washing once, specifically twice or three times at a salt concentration and a temperature corresponding to 60° C., 1×SSC, 0.1% SDS, specifically 60° C., 0.1×SSC, 0.1% SDS, and more specifically 68° C., 0.1×SSC, 0.1% SDS. Hybridization requires that two polynucleotides have complementary sequences, although mismatches between bases are possible depending on the stringency of the hybridization. The term “complementary” is used to describe the relationship between nucleotide bases that may hybridize with each other. For example, with respect to DNA, adenosine is complementary to thymine, and cytosine is complementary to guanine. Therefore, the polynucleotide of the present disclosure may include isolated polynucleotide fragments complementary to the entire sequence as well as polynucleotide sequences substantially similar thereto.

Specifically, the polynucleotides having a homology or identity may be detected using the hybridization conditions including a hybridization step at a Tₘ value of 55° C. under the above-described conditions. Further, the Tₘ value may be 60° C., 63° C., or 65° C., but is not limited thereto, and may be appropriately adjusted by those skilled in the art depending on the purpose thereof.

As used herein, the term “homology” or “identity” refers to a degree of relatedness between two given amino acid sequences or nucleotide sequences, and may be expressed as a percentage. The terms homology and identity may be often used interchangeably with each other. The sequence homology or identity of conserved polynucleotide or polypeptide sequences may be determined by standard alignment algorithms and may be used with a default gap penalty established by the program being used. Substantially, homologous or identical sequences are generally expected to hybridize to all or at least about 50%, 60%, 70%, 80%, or 90% of the entire length of the sequences under moderate or highly stringent conditions. Polynucleotides that contain degenerate codons instead of codons in hybridizing polynucleotides are also considered.

The homology or identity of the polypeptide or polynucleotide sequences may be determined by, for example, the BLAST algorithm according to the literature (see Karlin and Altschul, Pro. Natl. Acad. Sci. USA, 90, 5873 (1993)), or FASTA by Pearson (see Methods Enzymol., 183, 63, 1990). Based on the algorithm BLAST, a program referred to as BLASTN or BLASTX has been developed (see http://www.ncbi.nlm.nih.gov). Further, whether any amino acid or polynucleotide sequences have homology, similarity, or identity with each other may be identified by comparing the sequences in a Southern hybridization experiment under stringent conditions as defined, and appropriate hybridization conditions defined are within the skill of the art, and may be determined by a method well known to those skilled in the art (e.g., J. Sambrook et al., Molecular Cloning, A Laboratory Manual, 2nd Edition, Cold Spring Harbor Laboratory Press, Cold Spring Harbor, N.Y. 1989; F. M. Ausubel et al., Current Protocols in Molecular Biology, John Wiley & Sons, Inc., New York).

As used herein, the term “enhancement of protein activity” means that the activity is enhanced as compared to the endogenous activity possessed by a microorganism or the activity before transformation. The enhancement of activity may include both introducing a foreign protein and enhancing the activity of an endogenous protein. That is, it includes introducing a foreign protein into a microorganism having intrinsic activity of a specific protein, and introducing the protein into a microorganism having no intrinsic activity. The “introduction of the protein” means that activity of a specific protein is introduced into a microorganism such that the protein activity is modified for expression. It may also be expressed as the enhancement of the activity of the corresponding protein.

As used herein, the term “endogenous” refers to a state originally possessed by a parent strain prior to transformation, when the traits of the microorganism are altered by way of genetic modification due to natural or artificial factors.

In the present disclosure, the enhancement of activity may be performed by way of the following methods of:

1) increasing the copy number of the polynucleotide encoding the protein:

2) modifying an expression regulatory sequence such that the expression of the polynucleotide is increased;

3) modifying the polynucleotide sequence on a chromosome such that the activity of the protein is enhanced;

4) introducing a foreign polynucleotide exhibiting the activity of the protein or a modified polynucleotide in which the codons of the above polynucleotide have been optimized; or

5) modification to enhance the activity by way of a combination of the above methods, but the method is not limited thereto.

1) The increasing of the copy number of the polynucleotide may be performed in a form in which the polynucleotide is operably linked to a vector, or by inserting into a chromosome of a host cell, but is not particularly limited thereto. Specifically, it may be performed by operably linking the polynucleotide encoding the protein of the present disclosure to a vector which may replicate and function regardless of the host cell, and then introducing the same into the host cell. Alternatively, it may be performed by way of a method of increasing the copy number of the polynucleotide in the chromosome of the host cell by operably linking the polynucleotide to a vector which may insert the polynucleotide into the chromosome of the host cell, and introducing the same into the host cell.

Next, 2) the modification of an expression regulatory sequence such that the expression of the polynucleotide is increased may be performed by inducing a modification in the sequence through deletion, insertion, or non-conservative or conservative substitution of a nucleic acid sequence, or a combination thereof so as to further enhance the activity of the expression regulatory sequence, or by replacing with a nucleic acid sequence having a stronger activity, but is not particularly limited thereto. The expression regulatory sequence may include a promoter, an operator sequence, a sequence encoding a ribosome binding domain, a sequence regulating the termination of transcription and translation, etc., but is not particularly limited thereto.

A strong heterologous promoter may be linked to the upstream region of the expression unit of the polynucleotide instead of the original promoter. Examples of the strong promoter include CJ7 promoter (Korean Patent No. 0620092 and WO 20061065095), lysCP1 promoter (WO 2009/096689), EF-Tu promoter, groEL promoter, aceA or aceB promoter, etc., but the strong promoter is not limited thereto. Further, 3) the modification of the polynucleotide sequence on a chromosome may be performed by inducing a modification in the expression regulatory sequence through deletion, insertion, or non-conservative or conservative substitution of a nucleic acid sequence, or a combination thereof so as to further enhance the activity of the polynucleotide sequence, or by replacing the polynucleotide sequence with a polynucleotide sequence modified to have a stronger activity, but is not particularly limited thereto.

Additionally, 4) the introduction a foreign polynucleotide sequence may be performed by introducing, into a host cell, a foreign polynucleotide encoding a protein that exhibits an activity identical or similar to that of the protein above, or a modified polynucleotide in which the codons of the foreign polynucleotide have been optimized. The foreign polynucleotide may be used without limitation to its origin or sequence as long as it exhibits an activity identical or similar to that of the protein. Further, the foreign polynucleotide may be introduced into a host cell after optimization of its codons so as to achieve the optimized transcription and translation in the host cell. The introduction may be performed by those skilled in the art by appropriately selecting a known transformation method, and a protein may be produced as the introduced polynucleotides are expressed in the host cell, thereby increasing its activity.

Finally, 5) the method of modification to enhance the activity by way of a combination of 1) to 4) may be performed by way of a combined application of one or more of the following methods: increasing the copy number of the polynucleotide encoding the protein; modifying an expression regulatory sequence such that the expression of the polynucleotide is increased: modifying the polynucleotide sequence on a chromosome: and modifying a foreign polynucleotide exhibiting the activity of the protein or a codon-optimized modified polynucleotide thereof.

As used herein, the term “vector” refers to a DNA construct containing the polynucleotide sequence encoding the target protein, which is operably linked to a suitable regulatory sequence such that the target protein may be expressed in an appropriate host. The regulatory sequence includes a promoter capable of initiating transcription, any operator sequence for the control of the transcription, a sequence encoding an appropriate mRNA ribosome binding domain, and a sequence regulating the termination of transcription and translation. After being transformed into a suitable host cell, the vector may be replicated or function irrespective of the host genome, and may be integrated into the host genome itself. For example, a polynucleotide encoding a target protein in the chromosome may be replaced with a modified polynucleotide through a vector for chromosomal insertion. The insertion of the polynucleotide into the chromosome may be performed by way of any method known in the art, for example, homologous recombination, but is not limited thereto.

The vector used in the present disclosure is not particularly limited, and any vector known in the art may be used. Examples of commonly used vectors may include natural or recombinant plasmids, cosmids, viruses, and bacteriophages. For example, as a phage vector or cosmid vector, pWE15, M13, MBL3, MBL4, IXII, ASHII, APII, t10, t11, Charon4A, Charon21A, etc. may be used, and as a plasmid vector, those based on pBR, pUC, pBluescriptll, pGEM, pTZ, pCL, pET, etc. may be used. Specifically, the vectors pDZ, pACYC177, pACYC184, pCL, pECCG117, pUC19, pBR322, pMW118, pCC1BAC, etc. may be used.

As used herein, the term “transformation” refers to a process of introducing a vector including a polynucleotide encoding a target polypeptide into a host cell, thereby enabling expression of the polypeptide encoded by the polynucleotide in the host cell. As long as the transformed polynucleotide may be expressed in the host cell, it does not matter whether it is inserted into the chromosome of a host cell and located therein or located outside the chromosome, and both cases may be included. Additionally, the polynucleotide includes DNA and RNA which encode the target protein. The polynucleotide may be introduced in any form as long as it may be introduced into a host cell and expressed therein. For example, the polynucleotide may be introduced into a host cell in the form of an expression cassette, which is a gene construct including all elements necessary for self-expression. The expression cassette may generally include a promoter operably linked to the polynucleotide, a transcription terminator, a ribosome binding domain, and a translation terminator. The expression cassette may be in the form of an expression vector capable of self-replication. Additionally, the polynucleotide may be introduced as it is into a host cell and operably linked to a sequence necessary for its expression in the host cell, but is not limited thereto.

Further, as used herein, the term “operably linked” refers to a functional linkage between the above gene sequence and a promoter sequence which initiates and mediates the transcription of the polynucleotide encoding the target protein of the present disclosure.

The method of transforming the vector of the present disclosure includes any method of introducing a nucleic acid into a cell, and may be performed by selecting a suitable standard technique as known in the art depending on the host cell. For example, the method may include electroporation, calcium phosphate (CaPO₄) precipitation, calcium chloride (CaCl₂) precipitation, microinjection, a polyethylene glycol (PEG) technique, a DEAE-dextran technique, a cationic liposome technique, a lithium acetate-DMSO technique, etc., but is not limited thereto.

As used herein, the term “microorganism for producing L-histidine” includes all wild-type microorganisms, or naturally or artificially genetically modified microorganisms, and it may refer to a microorganism naturally having the L-histidine producing ability, or a microorganism prepared by imparting the L-histidine producing ability to a parent strain without the L-histidine producing ability. Additionally, it may be a microorganism in which a particular mechanism is weakened or enhanced due to insertion of a foreign gene, or enhancement or inactivation of the activity of an endogenous gene, and it may be a microorganism in which genetic mutation occurs or activity is enhanced for the production of the desired L-histidine.

For example, the microorganism for producing L-histidine may be a microorganism having an enhanced glycine transporter activity. Alternatively, it may be a microorganism in which feedback of an enzyme on the histidine biosynthesis pathway is inhibited, a microorganism that produces histidine by enhancing or inhibiting an enzyme involved in the histidine biosynthesis pathway, or a microorganism that produces histidine by inactivating the activity of an enzyme or protein that does not affect histidine biosynthesis, thereby facilitating the metabolism of the histidine biosynthesis pathway.

Specifically, it may be a microorganism in which the activity of CycA protein is enhanced, or additionally, the feedback inhibition on the histidine biosynthesis pathway is inhibited by modifying a HisG polypeptide, or expression of one or more of genes encoding the enzyme group in the histidine biosynthesis pathway, including hisE, hisG, hisA, hisF, hisI, hisD, hisC, hisB, and hisN, is enhanced. Further, the microorganism may be a microorganism in which the enzyme in the histidine degradation pathway is inactivated, the activity of an intermediate, a cofactor, or a protein or enzyme on a pathway that consumes an energy source on the histidine biosynthesis pathway is inactivated, or a protein importing the target histidine is inactivated. For example, the microorganism may be a microorganism in which gamma-aminobutyrate permease (NCgl1108) is inactivated.

Additionally, the microorganism may be a microorganism in which activity of a protein or an enzyme not involved in the growth or histidine biosynthesis of the microorganism is inactivated. More specifically, the microorganism may be a microorganism in which activity of formyltetrahydrofolate deformylase (PurU) or transposase (NCgl2131) not involved in the growth or L-histidine biosynthesis of the microorganism is attenuated.

As used herein, the term “inactivation of protein activity” means that the enzyme or protein is not expressed, or it has no activity thereof or has decreased activity even though expressed, as compared to a natural wild-type strain, a parent strain, or a strain in which the corresponding protein is not modified. In this regard, the decrease is a comprehensive concept including the case where the protein activity itself is decreased as compared with the activity of the protein originally possessed by a microorganism due to the mutation of the gene encoding the protein, modification of the expression regulatory sequence, or deletion in a part or all of genes, etc.; the case where the overall level of intracellular protein activity is decreased as compared with that of a natural strain or a strain before modification due to the inhibition of expression of the gene encoding the protein or the inhibition of translation; and a combination thereof. In the present disclosure, the inactivation may be achieved by applying various methods well known in the art. Examples of the methods may include 1) a method of deleting a part or all of the gene encoding the protein; 2) a method of modifying the expression regulatory sequence such that the expression of the gene encoding the protein is decreased; 3) a method of modifying the gene sequence encoding the protein such that the protein activity is removed or weakened; 4) a method of introducing an antisense oligonucleotide (e.g., antisense RNA) that binds complementarily to the transcript of the gene encoding the protein; 5) a method of adding a complementary sequence to the Shine-Dalgarno sequence upstream of the Shine-Dalgarno sequence of the gene encoding the protein to form a secondary structure, thereby inhibiting the ribosomal attachment; and 6) a reverse transcription engineering (RTE) method of adding a promoter at the 3′ terminus of an open reading frame (ORF) of the polynucleotide sequence of the gene encoding the protein so as to be reversely transcribed; and a combination thereof, but is not particularly limited thereto.

However, this is merely an example, and the method is not limited thereto. Additionally, it may be a microorganism, in which the expression of genes encoding enzymes of various known L-histidine biosynthesis pathways is enhanced, enzymes on degradation pathways are inactivated, the activity of an intermediate, a cofactor, or an enzyme on a pathway that consumes an energy source on the histidine biosynthesis pathway is inactivated. The microorganism for producing L-histidine may be prepared by applying various known methods.

With respect to the objects of the present disclosure, the microorganism of the present disclosure may be any microorganism as long as it includes the glycine transporter and is capable of producing L-histidine.

As used herein, the term “microorganism capable of producing L-histidine” may be interchangeably used with “microorganism producing L-histidine”, “microorganism having an L-histidine producing ability”, and “microorganism for producing L-histidine”.

The microorganism producing histidine of the present disclosure may be a microorganism in which the activity of the glycine cleavage protein is further enhanced. The “microorganism producing histidine” and “enhancement of protein activity” are the same as described above.

As used herein, the term “glycine cleavage protein” is a protein that is directly or indirectly involved in the glycine cleavage pathway, and may be used to mean each protein constituting the “glycine cleavage system (GCV)”, or the complex of the proteins, or the glycine cleavage system itself.

Specifically, the glycine cleavage protein may be any one or more selected from the group consisting of T-protein (GcvT), P-protein (GcvP), L-protein (GcvL), H-protein (GcvH) that constitute the glycine cleavage system, and LipB or LipA, which are coenzymes of the glycine cleavage system, but is not limited thereto (John E. Cronan, Microbiology and Molecular Biology Reviews, 13 Apr. 2016). The glycine cleavage protein may be derived from a microorganism of the genus Corynebacterium, specifically Corynebacterium ammoniagenes, but is not limited thereto. The GcvP protein may include an amino acid sequence of SEQ ID NO: 26, the GcvT protein may include an amino acid sequence of SEQ ID NO: 27, the GcvH protein may include an amino acid sequence of SEQ ID NO: 28, the LipA protein may include an amino acid sequence of SEQ ID NO: 29, and the LipB protein may include an amino acid sequence of SEQ ID NO: 30, or each may include an amino acid sequence having 70% or more homology to the respective amino acid sequence, but is not limited thereto. Specifically, the GcvP protein may include the amino acid sequence of SEQ ID NO: 26, or an amino acid sequence having at least 70%, 75%, 80%, 85%, 86%, 87%, 88%, 89%, 90%, 91%, 92%, 93%, 94%, 95%, 96%, 97%, 98%, or 99% homology or identity to the amino acid sequence of SEQ ID NO: 26. The description of homology or identity is the same for GcvT, GcvH, LipA, and LipB. Additionally, it is apparent that any protein having an amino acid sequence in which part of the amino acid sequence is deleted, modified, substituted, or added may also fall within the scope of the present disclosure as long as the amino acid has such a homology or identity and exhibits efficacy corresponding to that of the above protein.

Further, a probe that may be prepared from a known gene sequence, for example, any polypeptide having a glycine cleavage activity as a polypeptide encoded by a polynucleotide which may hybridize with a sequence complementary to all or part of the nucleotide sequence encoding the polypeptide under stringent conditions, may be included without limitation.

The homology or identity are as described above.

As used herein, the term “microorganism of the genus Corynebacterium for producing L-histidine” may refer to a microorganism that produces L-histidine and belongs to the genus Corynebacterium. The microorganism producing L-histidine is the same as described above. Specifically, in the present disclosure, the microorganism of the genus Corynebacterium having an L-histidine producing ability may refer to a microorganism of the genus Corynebacterium in which the activity of the glycine transporter of the present disclosure is enhanced, or which has been transformed with a vector containing the gene encoding the glycine transporter to have an enhanced L-histidine producing ability. Alternatively, it may refer to a microorganism of the genus Corynebacterium in which the activity of the glycine cleavage protein is further enhanced, or which has been transformed with a vector containing the gene encoding the glycine cleavage protein to have an enhanced L-histidine producing ability. The “microorganism of the genus Corynebacterium having an enhanced L-histidine producing ability” may refer to a microorganism in which the L-histidine producing ability is improved as compared with a parent strain before transformation or a non-modified microorganism. The ‘non-modified microorganism’ may refer to a natural strain of the genus Corynebacterium itself, a microorganism not containing the gene encoding the glycine transporter, or a microorganism that has not been transformed with a vector containing the gene encoding the glycine transporter.

As used herein, the term “microorganism of the genus Corynebacterium” may include all microorganisms of the genus Corynebacterium. Specifically, it may be Corynebacterium glutamicum, Corynebacterium crudilactis, Corynebacterium deserti, Corynebacterium efficiens, Corynebacterium callunae, Corynebacterium stationis, Corynebacterium singulare, Corynebacterium halotolerans, Corynebacterium striatum, Corynebacterium ammoniagenes, Corynebacterium pollutisoli, Corynebacterium imitans, Corynebacterium testudinois, or Corynebacterium flavescens, and more specifically Corynebacterium glutamicum.

Another aspect of the present disclosure provides a composition for producing L-histidine, the composition including the microorganism for producing L-histidine of the present disclosure.

The composition for producing L-histidine may refer to a composition capable of producing L-histidine by the microorganism for producing L-histidine of the present disclosure. The composition may include the microorganism for producing L-histidine, and may include an additional composition capable of producing histidine using the strain without limitation. The additional component capable of producing histidine may further include, for example, any suitable excipient commonly used in a composition for fermentation, or components of a medium. Such excipients may be, for example, preservatives, wetting agents, dispersing agents, suspending agents, buffers, stabilizing agents, isotonic agents, etc., but are not limited thereto.

Still another aspect of the present disclosure provides use of the microorganism of the genus Corynebacterium having an enhanced glycine transporter activity in the production of L-histidine.

The “glycine transporter”, “enhancement of activity”, or “microorganism of the genus Corynebacterium” are as described above.

Still another aspect of the present disclosure provides a method of producing L-histidine, the method including the step of culturing the microorganism.

The medium and other culture conditions used for culturing the microorganism of the present disclosure may be any medium commonly used for culturing the microorganism of the genus Corynebacterium without any particular limitation. Specifically, the microorganism of the present disclosure may be cultured under aerobic or anaerobic conditions in a common medium containing an appropriate carbon source, nitrogen source, phosphorus source, inorganic compound, amino acid, and/or vitamin, etc., while adjusting temperature, pH, etc.

In the present disclosure, the carbon source may include carbohydrates, such as glucose, fructose, sucrose, maltose, etc.; alcohols, such as sugar alcohols, glycerol, etc.; fatty acids, such as palmitic acid, stearic acid, linoleic acid, etc.; organic acids, such as pyruvic acid, lactic acid, acetic acid, citric acid, etc.; amino acids, such as glutamic acid, methionine, lysine, etc., but is not limited thereto. Additionally, the carbon source may include natural organic nutrients such as starch hydrolysate, molasses, blackstrap molasses, rice bran, cassava, sugar cane molasses, corn steep liquor, etc., and carbohydrates such as sterilized pretreated molasses (i.e., molasses converted to reducing sugar) may be used. In addition, various other carbon sources in an appropriate amount may be used without limitation. These carbon sources may be used alone or in combination of two or more kinds thereof.

The nitrogen source may include inorganic nitrogen sources, such as ammonia, ammonium sulfate, ammonium chloride, ammonium acetate, ammonium phosphate, ammonium carbonate, ammonium nitrate, etc.; amino acids, such as glutamic acid, methionine, glutamine, etc.; and organic nitrogen sources, such as peptone, NZ-amine, meat extract, yeast extract, malt extract, corn steep liquor, casein hydrolysate, fish or decomposition products thereof, defatted soybean cake or decomposition products thereof, etc. These nitrogen sources may be used alone or in combination of two or more kinds thereof, but are not limited thereto.

The phosphorus source may include monopotassium phosphate, dipotassium phosphate, or corresponding sodium-containing salts, etc. Examples of the inorganic compound may include sodium chloride, calcium chloride, iron chloride, magnesium sulfate, iron sulfate, manganese sulfate, calcium carbonate, etc.

Additionally, the medium may include vitamins and/or appropriate precursors, etc. These media or precursors may be added to a culture medium in a batch culture or continuous manner, but are not limited thereto.

In the present disclosure, the pH of a culture medium may be adjusted during the culture of the microorganism by adding a compound such as ammonium hydroxide, potassium hydroxide, ammonia, phosphoric acid, sulfuric acid, etc. to the culture medium in an appropriate manner. Additionally, during the culture, an antifoaming agent such as a fatty acid polyglycol ester may be added to prevent foam generation. In addition, oxygen or oxygen-containing gas may be injected into the culture medium in order to maintain an aerobic state of the culture medium; or no gas may be injected or nitrogen, hydrogen, or carbon dioxide gas may be injected into the culture medium in order to maintain an anaerobic or microaerobic state.

The temperature of the culture medium may be in a range of 25° C. to 40° C., and more specifically 28° C. to 37° C., but is not limited thereto. The culture may be continued until the useful materials are obtained in desired amounts, and specifically for 1 hour to 100 hours, but is not limited thereto.

The method of producing L-histidine may include a step of recovering L-histidine from one or more materials selected from the microorganism, the medium, the culture medium thereof, the supernatant of the culture medium, the extract of the culture medium, and the lysate of the microorganism, after the culturing step.

In the step of recovering, L-histidine, which is the target material, may be recovered from the culture solution using a suitable method known in the art according to the method of culturing the microorganism of the present disclosure, for example, a batch, continuous, or fed-batch culture method. For example, to recover L-histidine, methods, such as precipitation, centrifugation, filtration, chromatography, crystallization, etc., may be used. For example, a supernatant obtained by removing a biomass by centrifuging the culture medium at a low speed may be separated through ion-exchange chromatography, but is not limited thereto.

The step of recovering may include a purification process.

Hereinafter, the present disclosure will be described in more detail with reference to Examples and Experimental Examples. However, these Examples and Experimental Examples are for illustrative purposes only, and the scope of the present disclosure is not intended to be limited by these Examples and Experimental Examples.

### Example 1. Preparation of Artificial Mutant Strain Having High Histidine Producing Ability

To obtain an artificial mutant strain having high L-histidine producing ability, a mutation in a microorganism was induced by way of the following method.

In detail, KCCM11795P (Korean Patent Application No. 10-2016-0030092), a histidine-producing strain prepared from Corynebacterium glutamicum ATCC13032 by treatment with NTG, was used to obtain a mutant strain. KCCM11795P strain activated by culturing in an activation medium for 16 hours was seeded in a seed medium and cultured for 14 hours. 5 mL of the culture medium was recovered. The recovered culture medium was washed with 100 mM citric buffer, and then N-methyl-N′-nitro-N-nitrosoguanidine (NTG) was added to a concentration of 200 mg/L and treated for 20 minutes, followed by washing with 100 mM phosphate buffer. A mortality rate was calculated by smearing the NTG-treated strain on a minimal medium, and as a result, the mortality rate was 85%.

To obtain a variant having resistance to 1,2,4-triazole-3-alanine (TRA), which is a derivative of L-histidine, the NTG-treated strain was smeared on a minimal medium to which 1,2,4-triazole-3-alanine was added at a concentration of 0.2 g/L, 0.5 g/L, or 1 g/L, and cultured at 30° C. for 5 days. Thus, among the variants found at the three concentrations, an artificial mutant strain having 1,2,4-triazole-3-alanine resistance and the highest histidine producing ability was obtained, and this strain was named as CA14-0682.

<Activation Medium>

1% beef extract, 1% polypeptone, 0.5% sodium chloride, 1% yeast extract, 2% agar, pH 7.2

<Seed Medium>

5% glucose, 1% bactopeptone, 0.25% sodium chloride, 1% yeast extract, 0.4% urea, pH 7.2

<Minimal Medium>

1.0% glucose, 0.4% ammonium sulfate, 0.04% magnesium sulfate, 0.1% potassium dihydrogen phosphate, 0.1% urea, 0.001% thiamine, 200 μg/L biotin, 2% agar, pH 7.0

To examine the L-histidine producing ability and L-glycine production amount of the selected CA14-0682 strain, the strain was cultured by way of the following method. Each strain was seeded into a 250 mL corner-baffle flask containing 25 mL of the seed medium and cultured at 30° C. for 20 hours with shaking at 200 rpm. Then, 1 mL of the seed culture solution was seeded into a 250 mL corner-baffle flask containing 25 mL of a production medium and cultured at 30° C. for 24 hours with shaking at 200 rpm. After completion of the culture, the production amounts of L-histidine and L-glycine were measured via HPLC.

<Production Medium>

5% glucose, 2% ammonium sulfate, 0.1% potassium dihydrogen phosphate, 0.05% magnesium sulfate heptahydrate, 2.0% corn steep liquor (CSL), 200 μg/L biotin, calcium carbonate, pH 7.2.

TABLE 1
Production amounts of L-histidine
and L-glycine of CA14-0682 strain

| | | Consumed | Production | Production |
|---|---|---|---|---|
| | | glucose | amount of | amount of |
| | OD | (g/L) | histidine (g/L) | glycine (g/L) |
| KCCM11795P | 110.2 | 100 | 2.99 | 1.41 |
| CA14-0682 | 50.1 | 100 | 14.25 | 6.99 |

The culture results showed that the artificial mutant strain CA14-0682 having resistance to high concentration of TRA had the L-histidine production ability of a yield of about 15%.

The CA14-0682 strain was deposited at the Korean Culture Center of Microorganisms (KCCM) and assigned Accession No. KCCM 80179.

### Example 2. Preparation of Vector for Introducing Corynebacterium ammoniagenes-Derived Glycine Transporter (CycA(Cam))

In order to insert a gene cycA (hereinafter referred to as cycA(cam), SEQ ID NO: 2) encoding a Corynebacterium ammoniagenes-derived CycA protein (hereinafter referred to as CycA(Cam), SEQ ID NO: 1) into the chromosome of Corynebacterium glutamicum, purU in Corynebacterium glutamicum was used as an insertion site (Journal of Biotechnology 104, 5-25 Jorn Kalinowski et al., 2003). In order to prepare vectors for purU deletion and target gene insertion, PCR was performed using the chromosome of ATCC13032 as a template and primer pairs of SEQ ID NOS: 3 and 4 and SEQ ID NOS: 5 and 6, respectively. PfuUltra™ high-fidelity DNA polymerase (Stratagene) was used as a polymerase for the PCR reaction. PCR conditions were as follows: 28 cycles of denaturation at 95° C. for 30 seconds; annealing at 55° C. for 30 seconds; and polymerization at 72° C. for 2 minutes, followed by polymerization at 72° C. for 5 minutes. As a result, DNA fragments of 1606 bp for del-purU (SEQ ID NO: 7) and 1625 bp for del-purU (SEQ ID NO: 8) were each obtained. The DNA products thus obtained were purified using a PCR purification kit (QIAGEN) and cloned into a pDZ (Korean Patent No. 10-0924065) vector using an Infusion Cloning Kit (TaKaRa) to prepare pDZΔpurU, which is a vector for purU deletion and target gene insertion.

In order to obtain a cycA(Cam) DNA fragment linked with a promoter (hereinafter referred to as Pn-cycA(Cam)), PCR was performed using the chromosome of Corynebacterium ammoniagenes ATCC6872 as a template and a primer pair of SEQ ID NOS: 9 and 10. PfuUltra™ high-fidelity DNA polymerase (Stratagene) was used as a polymerase for the PCR reaction. PCR conditions were as follows: 28 cycles of denaturation at 95° C. for 30 seconds; annealing at 55° C. for 30 seconds; and polymerization at 72° C. for 90 seconds, followed by polymerization at 72° C. for 5 minutes. As a result, a 1970 bp Pn-cycA(cam) DNA fragment was obtained. This amplified product was purified using a PCR purification kit (QIAGEN) and used as an insert DNA fragment for the preparation of a vector (SEQ ID NO: 11). The obtained DNA product was purified using a PCR purification kit (QIAGEN) and cloned into the prepared pDZΔpurU vector using an Infusion Cloning Kit (TaKaRa) to prepare pDZΔpurU::Pn-cycA(Cam), which is a cycA(Cam)-introduced vector.

### Example 3. Preparation of Strain Introduced with CA14-0682 Strain-Derived Glycine Transporter and Evaluation of Histidine Producing Ability

The vector pDZΔpurU::Pn-cycA(cam) prepared in Example 2 was transformed into CA14-0682 strain and subjected to secondary crossover to thereby prepare a strain in which purU gene on the chromosome was substituted with Pn-cycA(cam). This strain was named as CA14-0682ΔpurU::Pn-cycA(cam).

To examine L-histidine producing ability and L-glycine production amount of the prepared CA14-0682ΔpurU strain and CA14-0682ΔpurU::Pn-cycA(Cam) strain, these were cultured in the same manner as in Example 1.

TABLE 2
Production amounts of L-histidine and L-glycine
of strain introduced with CA14-0682-derived cycA(cam)

| | | Consumed | Producton | Producton |
|---|---|---|---|---|
| | | glucose | amount of | amount of |
| | OD | (g/L) | histidine (g/L) | glycine (g/L) |
| CA14-0682 | 50.2 | 100 | 14.85 | 7.41 |
| CA14-0682ΔpurU | 50.1 | 100 | 14.88 | 7.42 |
| CA14-0682ΔpurU::Pn-cycA(Cam) | 49.7 | 100 | 15.49 | 6.51 |

As a result of the evaluation, the parent strain CA14-0682 showed L-histidine production of 14.85 g/L and L-glycine production of 7.41 g/L. and the purU-deleted strain showed an L-histidine producing ability equivalent to that of the parent strain. In contrast, CA14-0682ΔpurU::Pn-cycA(Cam) strain showed a 4.3% increase in L-histidine production and a 13.8% decrease in L-glycine production. Therefore, it was confirmed that when extracellular L-glycine is imported into cells through introduction of the glycine transport gene, the L-histidine producing ability may be increased.

### Example 4. Preparation of CycA(Cam)-Overexpressing Recombinant Vector

In order to more strongly express intracellular cycA(Cam), a cycA(Cam)-overexpressing recombinant vector was prepared. pcj7 (Korean Patent No. 10-0620092), which is a known promoter derived from a microorganism of the Corynebacterium, and a known promoter (hereinafter referred to as PglyA) of glyA, which is a gene encoding serine hydroxymethyltransferase, were used.

In order to obtain a DNA fragment of the pcj7 promoter, PCR was performed using p117-cj7-gfp including pcj7 as a template. PfuUltra™ high-fidelity DNA polymerase (Stratagene) was used as a polymerase for the PCR reaction. The PCR reaction was performed using primers of SEQ ID NO: 12 and SEQ ID NO: 13 under the following conditions: 28 cycles of denaturation at 95° C. for 30 seconds; annealing at 55° C. for 30 seconds; and polymerization at 72° C. for 30 seconds, followed by polymerization at 72° C. for 1 minute. A PCR product thus amplified was purified using a PCR purification kit (QIAGEN) to obtain a pcj7 fragment of 350 bp.

In order to obtain a cycA(Cam) DNA fragment including a part of the pcj7 sequence in 5′, PCR was performed using the chromosome of Corynebacterium ammoniagenes ATCC6872 as a template. PfuUltra™ high-fidelity DNA polymerase (Stratagene) was used as a polymerase for the PCR reaction. The PCR reaction was performed using primers of SEQ ID NO: 14 and SEQ ID NO: 10 under the following conditions: 28 cycles of denaturation at 95° C. for 30 seconds; annealing at 55° C. for 30 seconds; and polymerization at 72° C. for 30 seconds, followed by polymerization at 72° C. for 1 minute. A PCR product thus amplified was purified using a PCR purification kit (QIAGEN) to obtain a cycA(Cam) fragment of 1647 bp including a part of the pcj7 sequence in 5′.

Sewing PCR was performed using the pcj7 fragment and the cycA(Cam) fragment obtained as above as templates and primers of SEQ ID NO: 12 and SEQ ID NO: 10. The PCR reaction was performed under the following conditions: 28 cycles of denaturation at 95° C. for 30 seconds; annealing at 55° C. for 30 seconds; and polymerization at 72° C. for 2 minutes, followed by polymerization at 72° C. for 5 minutes. As a result, a pcj7-cycA(Cam) gene fragment of 1964 bp was obtained, and this amplification product was purified using a PCR Purification kit (QIAGEN) and used as an insert DNA fragment for vector preparation (SEQ ID NO: 15). The obtained DNA product was purified using a PCR Purification kit (QIAGEN), and then cloned into the prepared pDZΔpurU vector using an Infusion Cloning Kit (TaKaRa) to prepare pDZΔpurU::pcj7-cycA(Cam), which is a vector for replacing the purU gene with the pcj7-cycA(Cam) gene.

Further, in order to obtain a PglyA DNA fragment, PCR was performed using the chromosome of ATCC13032 as a template. PfuUltra™ high-fidelity DNA polymerase (Stratagene) was used as a polymerase for the PCR reaction. The PCR reaction was performed using primers of SEQ ID NO: 16 and SEQ ID NO: 17 under the following conditions: 28 cycles of denaturation at 95° C. for 30 seconds; annealing at 55° C. for 30 seconds; and polymerization at 72° C. for 30 seconds, followed by polymerization at 72° C. for 1 minute. A PCR product thus amplified was purified using a PCR purification kit (QIAGEN) to obtain a PglyA fragment of 340 bp.

In order to obtain a cycA(Cam) DNA fragment including a part of the PglyA sequence in 5′, PCR was performed using the chromosome of Corynebacterium ammoniagenes ATCC6872 as a template. PfuUltra™ high-fidelity DNA polymerase (Stratagene) was used as a polymerase for the PCR reaction. The PCR reaction was performed using primers of SEQ ID NO: 18 and SEQ ID NO: 10 under the following conditions: 28 cycles of denaturation at 95° C. for 30 seconds; annealing at 55° C. for 30 seconds; and polymerization at 72° C. for 30 seconds, followed by polymerization at 72° C. for 1 minute. A PCR product thus amplified was purified using a PCR purification kit (QIAGEN) to obtain a cycA(Cam) fragment of 1647 bp including a part of the PglyA sequence in 5′.

Sewing PCR was performed using the PglyA fragment and the cycA(Cam) fragment obtained as above as templates and primers of SEQ ID NO: 16 and SEQ ID NO: 10. The PCR reaction was performed under the following conditions: 28 cycles of denaturation at 95° C. for 30 seconds; annealing at 55° C. for 30 seconds; and polymerization at 72° C. for 2 minutes, followed by polymerization at 72° C. for 5 minutes. As a result, a PglyA-cycA(Cam) gene fragment of 1963 bp was obtained, and this amplification product was purified using a PCR Purification kit (QIAGEN) and used as an insert DNA fragment for vector preparation (SEQ ID NO: 19). The obtained DNA product was purified using a PCR Purification kit (QIAGEN), and then cloned into the prepared pDZΔpurU vector using an Infusion Cloning Kit (TaKaRa) to prepare pDZΔpurU::PglyA-cycA(Cam), which is a vector for replacing the purU gene with the PglyA-cycA(Cam) gene.

### Example 5. Preparation of Vector for Introducing E. coli-Derived Glycine Transporter (CycA(Eco))

Meanwhile, to compare Corynebacterium ammoniagenes-derived CycA protein and activity thereof, a vector was prepared for introducing pcj7 operably linked with cycA (hereinafter referred to as cycA(Eco), SEQ ID NO: 21), which is a gene encoding E. coli K-12-derived CycA protein previously disclosed (hereinafter referred to as CycA(Eco), SEQ ID NO: 20) (Microbiology, 141(Pt 1); 133-40, 1995).

To obtain a DNA fragment of pcj7 promoter, PCR was performed using p117-cj7-gfp including pcj7 as a template. PfuUltra™ high-fidelity DNA polymerase (Stratagene) was used as a polymerase for the PCR reaction. The PCR reaction was performed using primers of SEQ ID NO: 12 and SEQ ID NO: 22 under the following conditions: 28 cycles of denaturation at 95° C. for 30 seconds; annealing at 55° C. for 30 seconds; and polymerization at 72° C. for 30 seconds, followed by polymerization at 72° C. for 1 minute. A PCR product thus amplified was purified using a PCR purification kit (QIAGEN) to obtain a pcj7 fragment of 350 bp.

In order to obtain a cycA(Eco) DNA fragment including a part of the pcj7 sequence in 5′, PCR was performed using the chromosome of E. coli K-12 W3110 as a template and primers of SEQ ID NO: 23 and SEQ ID NO: 24. PfuUltra™ high-fidelity DNA polymerase (Stratagene) was used as a polymerase for the PCR reaction. The PCR conditions were as follows: 28 cycles of denaturation at 95° C. for 30 seconds; annealing at 55° C. for 30 seconds; and polymerization at 72° C. for 1 minute, followed by polymerization at 72° C. for 5 minutes. As a result, a cycA(Eco) gene fragment of 1659 bp was obtained, and this amplification product was purified using a PCR purification kit (QIAGEN) and used as an insert DNA fragment for vector preparation.

Sewing PCR was performed using the pcj7 fragment and the cycA(Eco) fragment as templates and primers of SEQ ID NO: 12 and SEQ ID NO: 24. The PCR reaction was performed under the following conditions: 28 cycles of denaturation at 95° C. for 30 seconds; annealing at 55° C. for 30 seconds; and polymerization at 72° C. for 90 seconds, followed by polymerization at 72° C. for 5 minutes. As a result, a pcj7-cycA(Eco) gene fragment of 1985 bp was obtained (SEQ ID NO: 25). This amplification product was purified using a PCR Purification kit (QIAGEN), and then cloned into the pDZΔpurU vector using an Infusion Cloning Kit (TaKaRa) in accordance with the provided manual to prepare pDZΔpurU::pcj7-cycA(Eco), which is a vector for replacing the purU gene with the pcj7-cycA(Eco) gene.

### Example 6. Preparation of CA14-0682-Derived cycA(Cam) or cycA(Eco)-Overexpressing Strain and Comparison of Histidine Producing Ability

In order to prepare a cycA(Cam) or cycA(Eco)-overexpressing strain using a CA14-0682 strain as a parent strain, the prepared four vectors (pDZΔpurU, pDZΔpurU::pcj7-cycA(Cam), pDZΔpurU::PglyA-cycA(Cam), and pDZΔpurU::pcj7-cycA(Eco)) were each transformed into the CA14-0682 strain via electroporation. Secondary crossover was performed to obtain a strain in which deletion of purU gene on the chromosome and substitution in the form of pcj7-cycA(Cam) or PglyA-cycA(Cam) or pcj7-cycA(Eco) were performed, respectively. Through this process, four strains (CA14-0682ΔpurU, CA14-0682ΔpurU::pcj7-cycA(Cam), CA14-0682ΔpurU::PglyA-cycA(Cam), and CA14-0682ΔpurU::pcj7-cycA(Eco)) were prepared.

In order to examine L-histidine producing ability and L-glycine production amount of the prepared four strains, the strains were each cultured in the same manner as in Example 1.

TABLE 3
Production amounts of L-histidine and L-glycine
of strain introduced with CA14-0682-derived cycA

| | | Consumed | Production | Production |
|---|---|---|---|---|
| | | glucose | amount of | amount of |
| | OD | (g/L) | histidine (g/L) | glycine (g/L) |
| CA14-0682 | 50.3 | 100 | 15.11 | 7.46 |
| CA14-0682ΔpurU | 50.5 | 100 | 15.05 | 7.42 |
| CA14-0682ΔpurU::pcj7_cycA(Cam) | 40.1 | 100 | 16.18 | 5.99 |
| CA14-0682ΔpurU::PglyA_cycA(Cam) | 44.7 | 100 | 16.14 | 5.89 |
| CA14-0682ΔpurU::pcj7_cycA(Eco) | 51.3 | 100 | 15.01 | 7.25 |

As a result of the evaluation, the CA14-0682ΔpurU::pcj7-cycA(Eco) strain introduced with E. coli-derived cycA showed rare uptake of Gly and L-histidine producing ability equivalent to or lower than that of the parent strain. In contrast, the CA14-0682ΔpurU::pcj7-cycA(Cam) strain and the CA14-0682ΔpurU::PglyA_cycA(Cam) strain, each enhanced by introduction with Corynebacterium ammoniagenes-derived cycA, showed a decrease in the Gly producing ability and 7.1% and 6.8% increase in histidine producing ability as compared with the parent strain, respectively. Therefore, it was confirmed that Corynebacterium ammoniagenes-derived cycA introduced into Corynebacterium glutamicum showed the high glycine transport ability to exhibit a high effect on the histidine producing ability due to the imported glycine as compared with E. coli-derived cycA. It was also confirmed that when cycA(Cam) is introduced and expressed through the glyA promoter, it is more beneficial in obtaining a cell mass. The CA14-0682ΔpurU::PglyA-cycA(Cam) strain was named as a CA14-0682-cycA(Cam) strain.

### Example 7. Preparation of Vector for Introducing Corynebacterium ammoniagenes-Derived Glycine Cleavage System

Since it had been previously confirmed that the activity of the glycine transporter had an effect on the increase in the histidine producing ability, the histidine producing ability was examined when the intracellular utilization of glycine imported into the cells was further increased. In detail, a glycine cleavage system (hereinafter referred to as a GCV system) was introduced. With regard to the Corynebacterium glutamicum strain, among the six proteins constituting the GCV system, only genes encoding L-proteins, such as LipB and LipA, are known, but genes encoding the other three proteins are not known. Therefore, in order to introduce the GCV system derived from Corynebacterium ammoniagenes, vectors for introducing genes (gcvP (SEQ ID NO: 31), gcvT (SEQ ID NO: 32), gcvH (SEQ ID NO: 33), lipA (SEQ ID NO: 34), and lipB (SEQ ID NO: 35)) encoding P-protein (SEQ ID NO: 26). T-protein (SEQ ID NO: 27), H-protein (SEQ ID NO: 28), LipA (SEQ ID NO: 29), and LipB (SEQ ID NO: 30)) were prepared. The genes form two pairs of operons (gcvP-gcvT and gcvH-lipB-lipA) on the chromosome of Corynebacterium ammoniagenes (hereinafter referred to as gcvPT and gcvH-lipBA). To introduce the GCV system, NCgl2131 gene among genes encoding transposon of Corynebacterium glutamicum was used as an insertion site (Journal of Biotechnology 104. 5-25 Jorn Kalinowski et al., 2003). In order to replace the NCgl2131 gene with the GCV system genes, a vector for NCgl2131 deletion and target gene insertion was prepared. To prepare the vector, PCR was performed using the chromosome of ATCC13032 as a template and primer pairs of SEQ ID NOS: 36 and 37 and SEQ ID NOS: 38 and 39, respectively. PfuUltra™ high-fidelity DNA polymerase (Stratagene) was used as a polymerase for the PCR reaction. The PCR conditions were as follows: 28 cycles of denaturation at 95° C. for 30 seconds; annealing at 55° C. for 30 seconds; and polymerization at 72° C. for 2 minutes, followed by polymerization at 72° C. for 5 minutes. As a result, DNA fragments of 531 bp for del-N2131L (SEQ ID NO: 40) and 555 bp for del-N2131R (SEQ ID NO: 41) were each obtained. The obtained DNA product was purified using a PCR purification kit (QIAGEN) and then cloned into the pDZ vector using an Infusion Cloning Kit (TaKaRa) to prepare pDZΔN2131, which is a vector for NCgl2131 gene deletion and target gene insertion.

In order to obtain a gcvPT gene fragment linked with a promoter (hereinafter referred to as Pn_gcvPT(cam)), PCR was performed using the chromosome of Corynebacterium ammoniagenes ATCC6872 as a template and primers of SEQ ID NO: 42 and SEQ ID NO: 43. PfuUltra™ high-fidelity DNA polymerase (Stratagene) was used as a polymerase for the PCR reaction. The PCR conditions were as follows: 28 cycles of denaturation at 95° C. for 30 seconds; annealing at 55° C. for 30 seconds; and polymerization at 72° C. for 5 minutes, followed by polymerization at 72° C. for 7 minutes. As a result, a Pn_gcvPT(Cam) gene fragment of 4499 bp including the promoter was obtained. This amplification product was purified using a PCR purification kit (QIAGEN) and used as an insert DNA fragment for vector preparation (SEQ ID NO: 44).

In order to obtain a gcvH-lipBA gene fragment linked with a promoter (hereinafter referred to as Pn_gcvH-lipBA(Cam)), PCR was performed using the chromosome of Corynebacterium ammoniagenes ATCC6872 as a template and primers of SEQ ID NO: 45 and SEQ ID NO: 46. PfuUltra™ high-fidelity DNA polymerase (Stratagene) was used as a polymerase for the PCR reaction. The PCR conditions were as follows: 28 cycles of denaturation at 95° C. for 30 seconds; annealing at 55° C. for 30 seconds; and polymerization at 72° C. for 1 minute, followed by polymerization at 72° C. for 7 minutes. As a result, a Pn_gcvH-lipBA(Cam) gene fragment of 3053 bp including the promoter was obtained. This amplification product was purified using a PCR purification kit (QIAGEN) and used as an insert DNA fragment for vector preparation (SEQ ID NO: 47).

Sewing PCR was performed using the Pn_gcvPT(Cam) fragment and the Pn_gcvH-lipBA(Cam) fragment obtained as above as templates and primers of SEQ ID NO: 42 and SEQ ID NO: 46. The PCR reaction was performed under the following conditions: 28 cycles of denaturation at 95° C. for 30 seconds; annealing at 55° C. for 30 seconds; and polymerization at 72° C. for 10 minutes, followed by polymerization at 72° C. for 12 minutes. As a result, a Pn_gcvPT(Cam)_Pn-gcvH-lipBA(Cam) gene fragment of 8259 bp was obtained, and this amplification product was purified using a PCR Purification kit (QIAGEN) and cloned into the prepared pDZΔN2131 vector using an Infusion Cloning Kit (TaKaRa) to prepare pDZΔN2131::GCV(Cam), which is a vector for replacing the NCgl2131 gene with the Pn_gcvPT(Cam)-Pn_gcvH-lipBA(Cam) gene.

### Example 8. Preparation of CA14-0682-Derived Strain Introduced with Glycine Cleavage System and Glycine Transporter and Evaluation of Histidine Producing Ability

The pDZΔN2131 and pDZΔN2131::GCV(Cam) vectors prepared in Example 7 were transformed into CA14-0682 strain and CA14-0682-cycA(Cam) strain, respectively. Then, secondary crossover was performed to prepare a strain (CA14-0682-cycA(Cam)ΔN2131), in which the NCgl2131 gene was deleted, a strain into which the glycine cleavage system alone was introduced, and two strains (CA14-0682ΔN2131::GCV(Cam) and CA14-0682-cycA(Cam)ΔN2131::GCV(Cam)) into which both of the glycine cleavage system and the glycine transporter were introduced. In order to examine L-histidine producing ability and L-glycine production amount of the prepared CA14-0682-cycA(Cam)ΔN2131 strain, CA14-0682ΔN2131::GCV(Cam) strain, and CA14-0682-cycA(Cam)ΔN2131::GCV(Cam) strain, these were cultured in the same manner as in Example 1, respectively.

TABLE 4
Production amounts of L-histidine and L-glycine of strain introduced
with CA14-0682-derived cycA(cam) and glycine cleavage system

| | | Consumed | Production | Production |
|---|---|---|---|---|
| | | glucose | amount of | amount of |
| | OD | (g/L) | histidine (g/L) | glycine (g/L) |
| CA14-0682 | 53.6 | 100 | 15.05 | 7.47 |
| CA14-0682-cycA(Cam)ΔN2131 | 45.1 | 100 | 16.19 | 6.68 |
| CA14-0682ΔN2131::GCV(Cam) | 48.9 | 100 | 16.52 | 4.72 |
| CA14-0682-cycA(Cam)ΔN2131::GCV(Cam) | 42.3 | 100 | 17.11 | 2.31 |

As a result of the evaluation, the CA14-0682-cycA(Cam)ΔN2131 strain, into which only the glycine transporter cycA(Cam) was introduced, showed a 7.6% increase in the histidine production amount and a 10.6% decrease in the glycine production amount as compared with the CA14-0682 strain, indicating that these results are equivalent to those of the CA14-0682ΔpurU::PglyA_cycA(Cam) (named as CA14-0682-cycA(Cam)) strain of Table 3. The CA14-0682ΔN2131::GCV(Cam) strain, into which the glycine cleavage system was introduced, showed a 9.8% increase in the histidine production amount and a 36.8% decrease in the glycine production amount as compared with the CA14-0682 strain. In contrast, the CA14-0682-cycA(Cam)ΔN2131::GCV(Cam) strain, prepared by additionally introducing GCV into the CA14-0682-cycA(Cam)ΔN2131 strain, showed a 13.7% increase in the histidine production amount and a 69.1% decrease in the glycine production amount as compared with the parent strain. Therefore, it was confirmed that although introduction of only the glycine transporter gene or glycine cleavage gene exhibits the effects of increasing histidine productivity and decreasing glycine productivity, introduction of both further increases histidine productivity with degradation of glycine produced in cells.

### Example 9. Preparation of Wild-Type Corynebacterium glutamicum-Derived Strain for Producing L-Histidine

Next, in order to examine the effects of introducing CycA and the GCV system into the wild-type Corynebacterium glutamicum strain, an L-histidine-producing strain was developed from the wild-type Corynebacterium glutamicum ATCC13032 strain.

### Example 9-1: Introduction of HisG Polypeptide Mutation

First, in order to release feedback inhibition of HisG polypeptide, which is a first enzyme in the L-histidine biosynthesis pathway, amino acids at positions 233 and 235 from the N-terminus of HisG were substituted from glycine to histidine (hereinafter referred to as G233H mutant) and from threonine to glutamine (hereinafter referred to as T235Q) (SEQ ID NO: 48) at the same time (ACS Synth. Biol., 2014, 3(1), pp. 21-29).

In detail, in order to prepare a vector for inserting a hisG polypeptide mutation, gene fragments of the upstream region (hereinafter referred to as G233H,T235Q-5′) of residues at positions 233 and 235 of the hisG polypeptide and the downstream region (hereinafter referred to as G233H,T235Q-3′) of residues at positions 233 and 235 of the hisG polypeptide were obtained by PCR using the chromosomal DNA of Corynebacterium glutamicum ATCC13032 as a template and primers of SEQ ID NOS: 49 and 50 and SEQ ID NOS: 51 and 52, respectively. Solg™ Pfu-X DNA polymerase was used as a polymerase. The PCR amplification conditions were as follows: denaturation at 95° C. for 5 minutes, 30 cycles of denaturation at 95° C. for 30 seconds; annealing at 60° C. for 30 seconds; and polymerization at 72° C. for 30 seconds, followed by polymerization at 72° C. for 5 minutes.

The amplified G233H,T235Q-5′ fragment and G233H,T235Q-3′ fragment were cloned into pDZ using a Gibson assembly method (D G Gibson et al, NATURE METHODS, VOL. 6 NO. 5, MAY 2009, NEBuilder HiFi DNA Assembly Master Mix) to prepare pDZ-hisG (G233H, T235Q), which is a vector introduced with hisG polypeptide mutation.

The prepared pDZ-hisG (G233H, T235Q) vector was transformed into a wild-type Corynebacterium glutamicum ATCC13032 strain by electroporation, and then secondary crossover was performed to obtain a strain in which amino acids at positions 233 and 235 of the HisG polypeptide were substituted from glycine to histidine and from threonine to glutamine on the chromosome. The corresponding genetic manipulation was identified by PCR using SEQ ID NO: 53 and SEQ ID NO: 54, which are able to amplify the outer region of the gene-inserted homologous recombination upstream and downstream regions, and by sequencing, and the strain was named as CA14-0011.

### Example 9-2: Enhancement of Histidine Biosynthesis Pathway

Next, to enhance the L-histidine biosynthesis pathway, biosynthetic genes separated into a total of four operons were prepared in the form of a promoter-substituted cluster, which was then introduced. In detail, biosynthetic genes separated into a total of four operons (hisE-hisG, hisA-impA-hisF-hisI, hisD-hisC-hisB, and cg0911-hisN) were operably linked to three known synthetic promoters (lysCP1 (Korean Patent No. 10-0930203), pcj7, or SPL13 (Korean Patent No. 10-1783170 B1)) or to gapA gene promoter, and respective operons were clustered and then introduced at once. Ncgl1108 gene encoding gamma-aminobutyrate permease was used as an insertion site (Microb Biotechnol. 2014 January; 7(1):5-25).

A specific experimental method is as follows. In order to prepare a vector for NCgl1108 gene deletion, gene fragments of the upstream region of NCgl1108 (hereinafter referred to as N1108-5′) and the downstream region of Ncgl1108 (hereinafter referred to as N1108-3′) were obtained by PCR using the chromosomal DNA of Corynebacterium glutamicum ATCC13032 as a template and primers of SEQ ID NOS: 55 and 56 and SEQ ID NOS: 57 and 58, respectively. Solg™ Pfu-X DNA polymerase was used as a polymerase. The PCR amplification conditions were as follows: denaturation at 95° C. for 5 minutes, 30 cycles of denaturation at 95° C. for 30 seconds; annealing at 60° C. for 30 seconds; and polymerization at 72° C. for 60 seconds, followed by polymerization at 72° C. for 5 minutes. The amplified N1108-5′ fragment and N1108-3′ fragment were cloned into pDZ using a Gibson assembly method to prepare a pDZΔN1108 vector, which is an NCgl1108-deleted vector.

The prepared pDZ-ΔNCgl1108 vector was transformed into a CA14-0011 strain by electroporation, and then secondary crossover was performed to obtain a strain in which the NCgl1108 gene on the chromosome was broken. The corresponding genetic manipulation was identified by PCR using SEQ ID NO: 59 and SEQ ID NO: 60, which are able to amplify the outer region of the gene-broken homologous recombination upstream and downstream regions, and by sequencing, and the strain was named as CA14-0736.

To enhance a histidine biosynthetic cluster, four operon gene clusters and promoter regions to be substituted were obtained. A lysCP1 promoter fragment and a hisE-hisG fragment, a gapA promoter fragment and a hisA-impA-hisF-hisI fragment, a SPL13 fragment and a hisD-hisC-hisB fragment, and a pcj7 fragment and a cg0911-hisN fragment were obtained.

To obtain the lysCP1 DNA fragment. PCR was performed using the chromosome of KCCM10919P strain (Korean Patent No. 10-0930203) as a template. PfuUltra™ high-fidelity DNA polymerase (Stratagene) was used as a polymerase for the PCR reaction. The PCR reaction was performed using primers of SEQ ID NO: 61 and SEQ ID NO: 62 under the following conditions: 28 cycles of denaturation at 95° C. for 30 seconds; annealing at 55° C. for 30 seconds; and polymerization at 72° C. for 30 seconds, followed by polymerization at 72° C. for 1 minute. The PCR product thus amplified was purified using a PCR Purification kit (QIAGEN) to obtain the lysCP1 fragment.

To obtain the hisE-hisG gene fragment, PCR was performed using the chromosome of CA14-0011 strain as a template. The PCR reaction was performed using primers of SEQ ID NO: 63 and SEQ ID NO: 64 under the following conditions: 28 cycles of denaturation at 95° C. for 30 seconds; annealing at 55° C. for 30 seconds; and polymerization at 72° C. for 2 minutes, followed by polymerization at 72° C. for 5 minutes. The PCR product thus amplified was purified using a PCR Purification kit (QIAGEN) to obtain the hisE-hisG fragment.

To obtain a promoter DNA fragment of Corynebacterium glutamicum-derived gapA gene (hereinafter referred to as PgapA), PCR was performed using the chromosome of Corynebacterium glutamicum ATCC13032 as a template. The PCR reaction was performed using primers of SEQ ID NO: 65 and SEQ ID NO: 66 under the following conditions: 28 cycles of denaturation at 95° C. for 30 seconds; annealing at 55° C. for 30 seconds; and polymerization at 72° C. for 2 minutes, followed by polymerization at 72° C. for 5 minutes. The PCR product thus amplified was purified using a PCR Purification kit (QIAGEN) to obtain the PgapA fragment.

To obtain a hisA-impA-hisF-hisI gene fragment, PCR was performed using the chromosome of CA14-0011 strain as a template. The PCR reaction was performed using primers of SEQ ID NO: 67 and SEQ ID NO: 68 under the following conditions: 28 cycles of denaturation at 95° C. for 30 seconds; annealing at 55° C. for 30 seconds; and polymerization at 72° C. for 2 minutes, followed by polymerization at 72° C. for 5 minutes. The PCR product thus amplified was purified using a PCR Purification kit (QIAGEN) to obtain the hisA-impA-hisF-hisI fragment.

To obtain a SPL13 DNA fragment, PCR was performed using SPL13 DNA as a template. The PCR reaction was performed using primers of SEQ ID NO: 69 and SEQ ID NO: 70 under the following conditions: 28 cycles of denaturation at 95° C. for 30 seconds; annealing at 55° C. for 30 seconds; and polymerization at 72° C. for 1 minute, followed by polymerization at 72° C. for 5 minutes. The PCR product thus amplified was purified using a PCR Purification kit (QIAGEN) to obtain the SPL13 DNA fragment.

To obtain a pcj7 promoter DNA fragment, PCR was performed using p117-cj7-gfp including pcj7 as a template. PfuUltra™ high-fidelity DNA polymerase (Stratagene) was used as a polymerase for the PCR reaction. The PCR reaction was performed using primers of SEQ ID NO: 71 and SEQ ID NO: 72 under the following conditions: 28 cycles of denaturation at 95° C. for 30 seconds; annealing at 55° C. for 30 seconds; and polymerization at 72° C. for 30 seconds, followed by polymerization at 72° C. for 1 minute. The PCR product thus amplified was purified using a PCR Purification kit (QIAGEN) to obtain the pcj7 fragment.

To obtain a hisD-hisC-hisB gene fragment, PCR was performed using the chromosome of CA14-0011 strain as a template. The PCR reaction was performed using primers of SEQ ID NO: 73 and SEQ ID NO: 74 under the following conditions: 28 cycles of denaturation at 95° C. for 30 seconds; annealing at 55° C. for 30 seconds; and polymerization at 72° C. for 5 minutes, followed by polymerization at 72° C. for 5 minutes. The PCR product thus amplified was purified using a PCR Purification kit (QIAGEN) to obtain the hisD-hisC-hisB gene fragment.

To obtain a cg0911-hisN gene fragment, PCR was performed using the chromosome of CA14-0011 strain as a template. The PCR reaction was performed using primers of SEQ ID NO: 75 and SEQ ID NO: 76 under the following conditions: 28 cycles of denaturation at 95° C. for 30 seconds; annealing at 55° C. for 30 seconds; and polymerization at 72° C. for 5 minutes, followed by polymerization at 72° C. for 5 minutes. The PCR product thus amplified was purified using a PCR Purification kit (QIAGEN) to obtain the cg0911-hisN gene fragment.

The obtained lysCP1 DNA fragment, hisE-hisG DNA fragment, PgapA DNA fragment, hisA-impA-hisF-hisI DNA fragment, SPL13 DNA fragment, hisD-hisC-hisB DNA fragment, pcj7 DNA fragment, and cg0911-hisN DNA fragment were cloned into a pDZ-ΔNcgl1108 vector using a Gibson assembly method to prepare pDZ-ΔNcgl1108::lysCP1_hisEG-PgapA_hisA-impA-hisFI-SPL13_HisDCB-pcj7_cg0911-hisN, which is a vector introduced with the L-histidine biosynthesis-enhanced cluster.

The prepared pDZ-ΔNcgl1108::lysCP1_hisEG-PgapA_hisA-impA-hisFI-SPL13_hisDCB-pcj7_cg0911-hisN vector was transformed into a CA14-0011 strain via electroporation, and then secondary crossover was performed to obtain a strain in which biosynthetic genes were inserted into the chromosome. The corresponding genetic manipulation was identified via PCR using SEQ ID NO: 59 and SEQ ID NO: 60, which are able to amplify the outer region of the gene-inserted homologous recombination upstream and downstream regions, and via genomic sequencing, and the strain was named as CA14-0737.

The CA14-0737 strain was deposited at the Korean Culture Center of Microorganisms (KCCM), an International Depositary Authority under the Budapest Treaty, on Nov. 27, 2018, and was assigned Accession No. KCCM12411P.

### Example 10. Preparation of Strain Introduced with CA14-0737 Strain-Derived Glycine Transporter and Glycine Cleavage System

The prepared four vectors (pDZΔpurU, pDZΔpurU::PglyA-cycA(Cam), pDZΔpurU::pcj7-cycA(Cam), and pDZΔpurU::pcj7-cycA(Eco)) were each transformed into CA14-0737 strain, and secondary crossover was performed to prepare a purU gene-deleted strain, a cycA(Cam)-introduced strain, and a cycA(Eco)-introduced strain, which were named as CA14-0737ΔpurU, CA14-0737ΔpurU::PglyA-cycA(Cam), CA14-0737ΔpurU::pcj7-cycA(Cam), and CA14-0737ΔpurU::pcj7-cycA(Eco). In order to examine L-histidine producing ability and L-glycine production amount of the prepared CA14-0737ΔpurU, CA14-0737ΔpurU::PglyA-cycA(Cam), CA14-0737ΔpurU::pcj7-cycA(Cam), and CA14-0737ΔpurU::pcj7-cycA(Eco) strains, these were cultured in the same manner as in Example 1.

TABLE 5
Production amounts of L-histidine and L-glycine
of strain introduced with CA14-0737-derived cycA

| | | Consumed | Production | Production |
|---|---|---|---|---|
| | | glucose | amount of | amount of |
| | OD | (g/L) | histidine (g/L) | glycine (g/L) |
| CA14-0737 | 88.4 | 100 | 4.11 | 2.21 |
| CA14-0737ΔpurU | 87.9 | 100 | 4.20 | 2.24 |
| CA14-0737ΔpurU::PglyA-cycA(Cam) | 87.4 | 100 | 4.93 | 1.90 |
| CA14-0737ΔpurU::pcj7-cycA(Cam) | 84.1 | 100 | 4.97 | 1.95 |
| CA14-0737ΔpurU::pcj7-cycA(Eco) | 88.9 | 100 | 4.29 | 2.20 |

As a result of the evaluation, the CA14-0737ΔpurU::pcj7-cycA(Eco) strain, into which E. coli-derived cycA was introduced, showed rare Gly uptake, and thus exhibited histidine producing ability equivalent to that of the parent strain. In contrast, the CA14-0737ΔpurU::pcj7-cycA(Cam) strain, into which the enhanced Corynebacterium ammoniagenes-derived cycA was introduced, showed a 20.9% increase in the histidine producing ability and a 11.8% decrease in the glycine production amount as compared with the parent strain. The strain in which cycA(Cam) was expressed through the glyA promoter also showed a 20% increase in the histidine producing ability and a 14% decrease in the glycine production amount. These results indicate that Corynebacterium ammoniagenes-derived cycA introduced into Corynebacterium glutamicum has higher glycine uptake to exhibit the higher effect of increasing histidine producing ability through the glycine than E. coli-derived cycA. Among these, the CA14-0737ΔpurU::PglyA-cycA(Cam) strain was named as CA14-0737-cycA(Cam).

The pDZΔN2131 and pDZΔN2131::GCV(Cam) vectors prepared in Example 7 were transformed into CA14-0737 strain and CA14-0737-cycA(Cam) strain, respectively. Then, secondary crossover was performed to prepare a strain (CA14-0737-cycA(Cam)ΔN2131), in which the NCgl2131 gene was deleted, a strain into which the glycine cleavage system alone was introduced, and two strains (CA14-0737ΔN2131::GCV(Cam), CA14-0737-cycA(Cam)ΔN2131::GCV(Cam)), into which both of the glycine cleavage system and the glycine transporter were introduced. In order to examine L-histidine producing ability and L-glycine production amount of the prepared CA14-0737-cycA(Cam)ΔN2131 strain. CA14-0737ΔN2131::GCV(Cam) strain, and CA14-0737-cycA(Cam)ΔN2131::GCV(Cam) strain, these were cultured in the same manner as in Example 1, respectively.

TABLE 6
Production amounts of L-histidine and L-glycine of strain introduced
with CA14-0737-derived cycA(cam) and glycine cleavage system

| | | Consumed | Production | Production |
|---|---|---|---|---|
| | | glucose | amount of | amount of |
| | OD | (g/L) | histidine (g/L) | glycine (g/L) |
| CA14-0737 | 88.1 | 100 | 4.15 | 2.17 |
| CA14-0737-cycA(Cam)ΔN2131 | 75.1 | 100 | 4.89 | 1.48 |
| CA14-0737ΔN2131::GCV(Cam) | 78.2 | 100 | 5.42 | 0.94 |
| CA14-0737-cycA(Cam)ΔN2131::GCV(Cam) | 71.3 | 100 | 5.97 | 0.46 |

As a result of the evaluation, the CA14-0737-cycA(Cam) ΔN2131 strain, into which only the glycine transporter cycA(Cam) was introduced, showed a 17.8% increase in the histidine production amount and a 13% decrease in the glycine production amount as compared with the parent strain. The CA14-0737-cycA(Cam)ΔN2131::GCV(Cam) strain, into which the glycine transporter cycA(cam) and GCV were introduced at the same time, showed a 43.9% increase in the histidine production amount and a 78.8% decrease in the glycine production amount as compared with the parent strain. The CA14-0737ΔN2131::GCV(Cam) strain, into which the glycine cleavage system was introduced, showed a 30.6% increase in the histidine production amount and a 56.7% decrease in the glycine production amount as compared with the parent strain, but glycine was still accumulated in the culture medium, and histidine productivity was also lower than that of the strain into which GCV was introduced together with cycA(Cam). Therefore, it was confirmed that although introduction of only the glycine transporter gene or glycine cleavage system exhibits the effects of increasing histidine productivity and decreasing glycine productivity, introduction of both of the glycine transporter and the glycine cleavage system further increases histidine productivity with degradation of glycine produced in cells. The CA14-0737-cycA(Cam) strain was named as CA14-0777, and the CA14-0737-cycA(Cam)ΔN2131::GCV(Cam) strain was named as CA14-0809. The two strains were deposited at the Korean Culture Center of Microorganisms (KCCM), an International Depositary Authority under the Budapest Treaty, on Apr. 15, 2019, and was assigned Accession Nos. KCCM12488P and KCCM12489P, respectively.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are therefore intended to be embraced by the claims.

### Effect of the Invention

A microorganism for producing L-histidine of the present disclosure may have an excellent histidine producing ability, and may thereby be applied to efficient mass production of L-histidine.

1. A microorganism of the genus Corynebacterium for producing L-histidine, the microorganism having an enhanced glycine transporter activity.

2. The microorganism of claim 1, wherein the glycine transporter is derived from Corynebacterium ammoniagenes.

3. The microorganism of claim 1, wherein the glycine transporter protein is a CycA protein.

4. The microorganism of claim 1, wherein the glycine transporter comprises an amino acid sequence of SEQ ID NO: 1 or an amino acid sequence having 90% or more sequence homology thereto.

5. The microorganism of claim 1, wherein activity of a glycine cleavage protein is further enhanced.

6. The microorganism of claim 5, wherein the glycine cleavage protein is one or more proteins selected from the group consisting of GcvP, GcvT, GcvH, LipB, and LipA.

7. The microorganism of claim 6, wherein the glycine cleavage protein is derived from Corynebacterium ammoniagenes.

8. The microorganism of claim 6, wherein the GcvP comprises an amino acid sequence of SEQ ID NO: 26, GcvT comprises an amino acid sequence of SEQ ID NO: 27, GcvH comprises an amino acid sequence of SEQ ID NO: 28, LipA comprises an amino acid sequence of SEQ ID NO: 29, and LipB comprises an amino acid sequence of SEQ ID NO: 30, or each comprises an amino acid sequence having 90% or more homology to the respective amino acid sequence.

9. The microorganism of claim 1, wherein the microorganism of the genus Corynebacterium for producing L-histidine is Corynebacterium glutamicum.

10. A composition for producing L-histidine, the composition comprising the microorganism of claim 1.

11. A method of producing L-histidine, the method comprising the steps of:
culturing the microorganism of claim 1 in a medium; and
recovering L-histidine from the microorganism and the medium.

12. Use of a microorganism of the genus Corynebacterium having an enhanced glycine transporter activity in the production of L-histidine.


SEQUENCE LISTING (76 sequences)

SEQ ID NO: 1 - protein, 476 aa, Corynebacterium ammoniagenes
MASYDPGHSQVKNTGVELDSHVESDHLQRGLSNRHIQLIAIGGAIGTGLFMGSGKTISLAGPSVILVYGIIGFMLFFVMRAMGELLLSNLNYKSLRDAVSDILGPTAGFVCGWTYWFCWIVTGMADVVAITGYTQYWWPDIALWIPGALTILLLLGLNLVAVRLFGELEFWFAIIKLVAITALILVGAVMVISRFQSPDGDIAAVSNLIDHGGFFPNGITGFLAGFQIAIFAFVGVELAGTAAAETKDPEKNLPRAINSIPIRIVVFYILALAVIMMVTPWDKVHSDSSPFVQMFALAGLPAAAGIINFVVLTSAASSANSGIFSTSRMLFGLAREGQAPKRWGILSRNQVPARGLLFSVACLVPSLAILYAGASVIDAFTLITTVSSVLFMVVWSLILAAYLVFRRKFPERHAASKFKVPGGAFMCWVVLAFFAFMVVVLTFENDTRSALMVTPIWFVILLAGWFISGGVKRARK

SEQ ID NO: 2 - DNA, 1431 bp, Corynebacterium ammoniagenes
atggcatcttatgatcccgggcattcacaggttaaaaatacaggagtagaactcgactctcacgtggagtcagaccatctccaacgcggtcttagcaaccgacatatccagttgattgccattggcggtgctatcggcaccggactatttatgggatcgggcaaaaccatctcccttgctggtccttccgtaatcttggtgtatggcatcatcggttttatgctcttcttcgtcatgcgggcgatgggtgagcttttgctctcgaacctcaactacaagtcattgcgtgatgccgtctcggacatcctcgggcctaccgctggtttcgtatgcggctggacctactggttctgctggattgtcacaggcatggctgatgtcgtggctattaccggctacacccaatactggtggccagatatcgcgctgtggatacctggagcattgacgattttattactgctcgggttaaatctcgtggcagtccgcctctttggtgagttggaattttggttcgcaatcatcaagctggtggctattactgcgctcatcctcgtcggcgcggtgatggttatttcgcgcttccaatccccagacggcgacattgcggctgtttccaacctgatcgaccatggcggcttcttcccgaacggaatcacgggcttcctcgccggattccagattgctatcttcgccttcgtcggcgtggagcttgccggtaccgcggctgcagaaaccaaagacccagaaaagaatcttcctcgcgcgattaactcgattcctattcgtatcgtcgtgttctacatcctggctcttgcggtcatcatgatggttaccccatgggacaaggtccacagtgactccagcccatttgtgcagatgtttgccctggctggactgccggcggcagcaggcattattaacttcgtggtgctgacatctgctgcttcatccgcgaacagtggtattttctccacatcacgcatgctgttcggtctggctcgcgaaggccaagcgcccaagcgttggggcatcttgtcccgtaaccaagtcccagcccgcggcctgctgttctcagtagcgtgcctggtcccgagcctggcaatcttgtacgccggcgccagcgtcattgacgcctttacgttgattaccaccgtgtcttctgtgctgttcatggtggtatggagcctgatcctcgcggcgtaccttgtcttccgccgcaagttcccagaacgccatgcagcatccaagttcaaggtcccaggcggggcgtttatgtgctgggttgttctcgctttcttcgcgtttatggtcgttgtactgacctttgaaaatgacactcggtccgctttgatggtcaccccaatctggttcgtgatcctcttggccggctggttcatctccggcggagtcaagcgcgctaggaagtga

SEQ ID NO: 3 - DNA, 40 bp, Artificial Sequence
  primer
agctcggtacccggggatccccaatgccacctgctgttcc

SEQ ID NO: 4 - DNA, 32 bp, Artificial Sequence
  primer
ctcgagactagttcaggcgcagcgctcggacg

SEQ ID NO: 5 - DNA, 32 bp, Artificial Sequence
  primer
actagtctcgagtggaggaccgggtgctggtt

SEQ ID NO: 6 - DNA, 40 bp, Artificial Sequence
  primer
ttgcatgcctgcaggtcgactgatggcctcccttgaccag

SEQ ID NO: 7 - DNA, 1606 bp, Artificial Sequence
  del-purU
agctcggtacccggggatccccaatgccacctgctgttccagaaccatcgcgggaaactcatccaacagcgcctgggcaacactcacgcagcgcctccccttcaccacatgcacctctggcattttgtggcgcacgacttttcccaaaaggacagtcacatggctcgctcccctcgggacgctaatttccgccgttcgaggttcatccaacagggagtatcctctcacaatcagtgcagtgaatcccctgattacaccgttgggaaatctccgcaatagcgcatctgccaccacaaccggattttccgcatattccttcttaatccaaaacgttcccactactcttgtgtactttgcagagaactgtcttctattcatttcatgctggacatcttcaaatctaattggcttcatgcttaagttagactgtgattccgccgattcggttccctcccgtttaaggcgcccaattctccaaatgagctctagtgtcagttcctcgccttcaagctcttaaaacggcacacaaagcatcaaatattttgcatgcaaggctatttttcagcccctttcaccccaccccaggagtctgtgagttccctgatcaactactcaccacaacgagcagcctcgcagcagaatcggtctgatttttgcatcccaaggcctctaaagcgggaaaatccaccacagatctcaacattcctgcaggttgtggtggatttttgcattgccccgaagcccgacccccgaatcgaacttcgaaccctcttaaccgccctcaattacccatcagtcccgattagaacgccaaactactccggcgcaccctccaaaatggctcccgcattagaagttccaatgcgagtagcccctgcttcaacaaacgccaccgcatcttcccaagttttcaccccaccggcagccttaattccaacccttccccgagaagccgaagccatcacccgaacagcctcaacagttgcccctcccgctgggtggaatcccgtggaagttttagcgaagtcagcaccagcagcaatcaacgcattcactgcagtcacaattgcttcatcactcacaacagctgtttcgaggatgaatttcagcaccacaggagatggaacagcctccctgattgccacaatttcctgcagcaacctattggcatctccctctttcactaccgcaatatccaaaacaacatctacttcggaagctccggactgtacggcaaggcgcgcttcggcggctttcaccaacgcgggggttttgccgtgcgggaatcctgcgacggtggccactcgaattccagcttcttgggcttttgcagttaggttgaccatgctgttggggacacagatcgtgccgactcccagctcaattgcggaatctataaatgcggccagttcggagttggttacttctggtccgaggagggtgtagtcaaggatttgggccatggtggagcgggaaatcgtcatatccataccctacttagacctgacttagtgtgggaaaatttccagggtagaatgcaacgaatgaccccgagttctcctgaagttcgtaatcgtccgagcgctgcgcctgaactagtctcgag

SEQ ID NO: 8 - DNA, 1625 bp, Artificial Sequence
  del-purU
actagtctcgagtggaggaccgggtgctggtttacggtaaccgcacggttgtctttgattaaggctttttgcttttcgacgcgaccctaaacaaatgagacctacccgagggtaggtctcatttgttttgtgtttagtctgtggtggtttcgcgtagtttttcttgcttaataaacagcaagagaagcagcgcgatgccgatcagtggcatcatcacgtagaacactgggatgagtgcgtcgttgtaggaaccggcgaacgcatcgtggagcgcggttggcaattgattgacgattgccggcgtcaattcgttggagtccagtccgccttgggctgccatggcggcttgttcttctggtgaaagttgtgccatggctgctggcattctttcttccatgagggttcccaagttgccaacgaacatgccaccgaccagcgcggatccgagtgaggaaccgatttgacggaagaagttgttcacagcggttgcggatccgaccaccgcggttggcagggtgttctgaacgatcaggaccagaacctgcatggctagacccaggccgacgccgaggacgaagaggtagattccgatctgccacagggtggtggacacttccattcgggcaaagaagatcagtgccacgaaggtaaccaccatgccgattggtgggaagagtttgtactttcctgtgttggagatgcggataccagtccagatggaggtacccatcatgccgaccatcattgggatcagcatgtagccggcttcggtggcgttgattccgtggaccatctggaggtaggtcggaaggtagccgatgatgccgaacattgcgatacccaggatcaggcctgcaatggtggtgagcgtgaagttgcggttttggaagaaggacattggaaccaatggatcttttgcgcgcagttccacaacaaccagcagtgcagcggcaacgatggtggtgatgatcagtccaatgatgattggatcagaccactcgtactgggatccaccccaggtggtgaacaggatcaggctggttgcggcaacgatcatgaagaaagtgcccaggtaatcccagcggaacttgacgctcttctttggaatgtccaggaagtaaatggcgacaccgatggcgatgattcccagtgggatgttcatccagaatgcccagcgccagcctggtccttcggtgaaccagccaccgagtagtgggccaagaactgcagagagtccgaagactccacccatgacacccatgtagcggccacgttcacgtgctggaacaacgtccgcgatgattgcctgcgagaggatcatcagtccaccgccaccgatgccctgtacggcacggccgacgatcaaccaggtcatatttcctgcaagcccgccgatgacggagccgatcaggaagatgccgagggctccgatgaagagacctttacgtccaaccaggtcgccgagctttccgtagatcggcagcatgatggtttcggcaagtaggtatgcggtgatgacccacatcatgtggtcaacgccgccgagttcaccgacgattgttggcagggctgtgccgaaaatcatctggtcaagggaggccatcagtcgacctgcaggcatgcaa

SEQ ID NO: 9 - DNA, 40 bp, Artificial Sequence
  primer
agcgctgcgcctgaactagttgccacggtgtttacttaca

SEQ ID NO: 10 - DNA, 40 bp, Artificial Sequence
  primer
gtcctccactcgagactagtactatctcgaagttgtcagc

SEQ ID NO: 11 - DNA, 1970 bp, Artificial Sequence
  Pn-cycA(cam)
agcgctgcgcctgaactagttgccacggtgtttacttacaacggcgatacaattgaaccgcaaatggctgctgagattgcggaactatcctgggtatcgccggatcagcctgatgttgtgcttgcgcctttgctcgctgattttgtctttcccgccattaattccaaggtgtcttgacggcactcttatctccgaacacgcgaagcctagtgtctccacaaaggggttggagagctagtggtagtttttctatcaaatagtaggtacgtgcgagagcgcgcagtgacgctctaccatttaagctcctactttcccattagcaagacaggtagattatggcatcttatgatcccgggcattcacaggttaaaaatacaggagtagaactcgactctcacgtggagtcagaccatctccaacgcggtcttagcaaccgacatatccagttgattgccattggcggtgctatcggcaccggactatttatgggatcgggcaaaaccatctcccttgctggtccttccgtaatcttggtgtatggcatcatcggttttatgctcttcttcgtcatgcgggcgatgggtgagcttttgctctcgaacctcaactacaagtcattgcgtgatgccgtctcggacatcctcgggcctaccgctggtttcgtatgcggctggacctactggttctgctggattgtcacaggcatggctgatgtcgtggctattaccggctacacccaatactggtggccagatatcgcgctgtggatacctggagcattgacgattttattactgctcgggttaaatctcgtggcagtccgcctctttggtgagttggaattttggttcgcaatcatcaagctggtggctattactgcgctcatcctcgtcggcgcggtgatggttatttcgcgcttccaatccccagacggcgacattgcggctgtttccaacctgatcgaccatggcggcttcttcccgaacggaatcacgggcttcctcgccggattccagattgctatcttcgccttcgtcggcgtggagcttgccggtaccgcggctgcagaaaccaaagacccagaaaagaatcttcctcgcgcgattaactcgattcctattcgtatcgtcgtgttctacatcctggctcttgcggtcatcatgatggttaccccatgggacaaggtccacagtgactccagcccatttgtgcagatgtttgccctggctggactgccggcggcagcaggcattattaacttcgtggtgctgacatctgctgcttcatccgcgaacagtggtattttctccacatcacgcatgctgttcggtctggctcgcgaaggccaagcgcccaagcgttggggcatcttgtcccgtaaccaagtcccagcccgcggcctgctgttctcagtagcgtgcctggtcccgagcctggcaatcttgtacgccggcgccagcgtcattgacgcctttacgttgattaccaccgtgtcttctgtgctgttcatggtggtatggagcctgatcctcgcggcgtaccttgtcttccgccgcaagttcccagaacgccatgcagcatccaagttcaaggtcccaggcggggcgtttatgtgctgggttgttctcgctttcttcgcgtttatggtcgttgtactgacctttgaaaatgacactcggtccgctttgatggtcaccccaatctggttcgtgatcctcttggccggctggttcatctccggcggagtcaagcgcgctaggaagtgagcgcaacaaagccccgccacagaaaaacgcgaggacttcgtgcattaagacgaaatatattagatgtccatgttgtctatcagacgggtgtcaccgacggagatagcgcccagtgctagagcaggagcggttggaggccacgcaatcggtttgagcgccttagggctgacaacttcgagatagtactagtctcgagtggaggac

SEQ ID NO: 12 - DNA, 40 bp, Artificial Sequence
  primer
agcgctgcgcctgaactagtagaaacatcccagcgctact

SEQ ID NO: 13 - DNA, 32 bp, Artificial Sequence
  primer
ataagatgccatgagtgtttcctttcgttggg

SEQ ID NO: 14 - DNA, 32 bp, Artificial Sequence
  primer
aaggaaacactcatggcatcttatgatcccgg

SEQ ID NO: 15 - DNA, 1964 bp, Artificial Sequence
  pcj7-cycA(Cam)
agcgctgcgcctgaactagtccagcgctactaatagggagcgttgaccttccttccacggaccggtaatcggagtgcctaaaaccgcatgcggcttaggctccaagataggttctgcgcggccgggtaatgcatcttctttagcaacaagttgaggggtaggtgcaaataagaacgacatagaaatcgtctcctttctgtttttaatcaacatacaccaccacctaaaaattccccgaccagcaagttcacagtattcgggcacaatatcgttgccaaaatattgtttcggaatatcatgggatacgtacccaacgaaaggaaacactcatggcatcttatgatcccgggcattcacaggttaaaaatacaggagtagaactcgactctcacgtggagtcagaccatctccaacgcggtcttagcaaccgacatatccagttgattgccattggcggtgctatcggcaccggactatttatgggatcgggcaaaaccatctcccttgctggtccttccgtaatcttggtgtatggcatcatcggttttatgctcttcttcgtcatgcgggcgatgggtgagcttttgctctcgaacctcaactacaagtcattgcgtgatgccgtctcggacatcctcgggcctaccgctggtttcgtatgcggctggacctactggttctgctggattgtcacaggcatggctgatgtcgtggctattaccggctacacccaatactggtggccagatatcgcgctgtggatacctggagcattgacgattttattactgctcgggttaaatctcgtggcagtccgcctctttggtgagttggaattttggttcgcaatcatcaagctggtggctattactgcgctcatcctcgtcggcgcggtgatggttatttcgcgcttccaatccccagacggcgacattgcggctgtttccaacctgatcgaccatggcggcttcttcccgaacggaatcacgggcttcctcgccggattccagattgctatcttcgccttcgtcggcgtggagcttgccggtaccgcggctgcagaaaccaaagacccagaaaagaatcttcctcgcgcgattaactcgattcctattcgtatcgtcgtgttctacatcctggctcttgcggtcatcatgatggttaccccatgggacaaggtccacagtgactccagcccatttgtgcagatgtttgccctggctggactgccggcggcagcaggcattattaacttcgtggtgctgacatctgctgcttcatccgcgaacagtggtattttctccacatcacgcatgctgttcggtctggctcgcgaaggccaagcgcccaagcgttggggcatcttgtcccgtaaccaagtcccagcccgcggcctgctgttctcagtagcgtgcctggtcccgagcctggcaatcttgtacgccggcgccagcgtcattgacgcctttacgttgattaccaccgtgtcttctgtgctgttcatggtggtatggagcctgatcctcgcggcgtaccttgtcttccgccgcaagttcccagaacgccatgcagcatccaagttcaaggtcccaggcggggcgtttatgtgctgggttgttctcgctttcttcgcgtttatggtcgttgtactgacctttgaaaatgacactcggtccgctttgatggtcaccccaatctggttcgtgatcctcttggccggctggttcatctccggcggagtcaagcgcgctaggaagtgagcgcaacaaagccccgccacagaaaaacgcgaggacttcgtgcattaagacgaaatatattagatgtccatgttgtctatcagacgggtgtcaccgacggagatagcgcccagtgctagagcaggagcggttggaggccacgcaatcggtttgagcgccttagggctgacaacttcgagatagtactagtctcgagtggaggac

SEQ ID NO: 16 - DNA, 40 bp, Artificial Sequence
  primer
agcgctgcgcctgaactagtaaatcaaggtaggggctgaa

SEQ ID NO: 17 - DNA, 30 bp, Artificial Sequence
  primer
ataagatgccatcaggtcagctaacctttc

SEQ ID NO: 18 - DNA, 30 bp, Artificial Sequence
  primer
ttagctgacctgatggcatcttatgatccc

SEQ ID NO: 19 - DNA, 1963 bp, Artificial Sequence
  PglyA-cycA(Cam)
agcgctgcgcctgaactagtaaatcaaggtaggggctgaaatctggtgtgcgtggggaaggtttcacaccagttgtgcttgcagcgttttgctctgccatgaatccattgtgcaccttagctactccactagtgtgatcggggttattttttcacttcaatgggtggctaaaagacgtgggcacgtgagtaaactcatgcgcgcgaaacgatgggagtgaacccatacttttatatatgggtatcggcggtctatgcttgtgggcgtacctgtcccgcgagtgaggtcttacgcgcgggattcgtcttgtgaaaggttagctgacctgatggcatcttatgatcccgggcattcacaggttaaaaatacaggagtagaactcgactctcacgtggagtcagaccatctccaacgcggtcttagcaaccgacatatccagttgattgccattggcggtgctatcggcaccggactatttatgggatcgggcaaaaccatctcccttgctggtccttccgtaatcttggtgtatggcatcatcggttttatgctcttcttcgtcatgcgggcgatgggtgagcttttgctctcgaacctcaactacaagtcattgcgtgatgccgtctcggacatcctcgggcctaccgctggtttcgtatgcggctggacctactggttctgctggattgtcacaggcatggctgatgtcgtggctattaccggctacacccaatactggtggccagatatcgcgctgtggatacctggagcattgacgattttattactgctcgggttaaatctcgtggcagtccgcctctttggtgagttggaattttggttcgcaatcatcaagctggtggctattactgcgctcatcctcgtcggcgcggtgatggttatttcgcgcttccaatccccagacggcgacattgcggctgtttccaacctgatcgaccatggcggcttcttcccgaacggaatcacgggcttcctcgccggattccagattgctatcttcgccttcgtcggcgtggagcttgccggtaccgcggctgcagaaaccaaagacccagaaaagaatcttcctcgcgcgattaactcgattcctattcgtatcgtcgtgttctacatcctggctcttgcggtcatcatgatggttaccccatgggacaaggtccacagtgactccagcccatttgtgcagatgtttgccctggctggactgccggcggcagcaggcattattaacttcgtggtgctgacatctgctgcttcatccgcgaacagtggtattttctccacatcacgcatgctgttcggtctggctcgcgaaggccaagcgcccaagcgttggggcatcttgtcccgtaaccaagtcccagcccgcggcctgctgttctcagtagcgtgcctggtcccgagcctggcaatcttgtacgccggcgccagcgtcattgacgcctttacgttgattaccaccgtgtcttctgtgctgttcatggtggtatggagcctgatcctcgcggcgtaccttgtcttccgccgcaagttcccagaacgccatgcagcatccaagttcaaggtcccaggcggggcgtttatgtgctgggttgttctcgctttcttcgcgtttatggtcgttgtactgacctttgaaaatgacactcggtccgctttgatggtcaccccaatctggttcgtgatcctcttggccggctggttcatctccggcggagtcaagcgcgctaggaagtgagcgcaacaaagccccgccacagaaaaacgcgaggacttcgtgcattaagacgaaatatattagatgtccatgttgtctatcagacgggtgtcaccgacggagatagcgcccagtgctagagcaggagcggttggaggccacgcaatcggtttgagcgccttagggctgacaacttcgagatagtactagtctcgagtggaggac

SEQ ID NO: 20 - protein, 470 aa, Escherichia coli
MVDQVKVVADDQAPAEQSLRRNLTNRHIQLIAIGGAIGTGLFMGSGKTISLAGPSIIFVYMIIGFMLFFVMRAMGELLLSNLEYKSFSDFASDLLGPWAGYFTGWTYWFCWVVTGMADVVAITAYAQFWFPDLSDWVASLAVIVLLLTLNLATVKMFGEMEFWFAMIKIVAIVSLIVVGLVMVAMHFQSPTGVEASFAHLWNDGGWFPKGLSGFFAGFQIAVFAFVGIELVGTTAAETKDPEKSLPRAINSIPIRIIMFYVFALIVIMSVTPWSSVVPEKSPFVELFVLVGLPAAASVINFVVLTSAASSANSGVFSTSRMLFGLAQEGVAPKAFAKLSKRAVPAKGLTFSCICLLGGVVMLYVNPSVIGAFTMITTVSAILFMFVWTIILCSYLVYRKQRPHLHEKSIYKMPLGKLMCWVCMAFFVFVVVLLTLEDDTRQALLVTPLWFIALGLGWLFIGKKRAAELRK

SEQ ID NO: 21 - DNA, 1413 bp, Escherichia coli
atggtagatcaggtaaaagtcgttgccgatgatcaggctccggctgaacagtcgctacggcgcaatctcacaaaccgacatattcagcttattgccattggcggtgccattggtacggggttgtttatggggtctggcaaaacgattagccttgccgggccgtcgatcattttcgtttatatgatcattggttttatgctctttttcgtgatgcgggcaatgggggaattgctgctttcgaatctggaatacaaatcttttagtgacttcgcttccgatttactcgggccgtgggcaggatatttcaccggctggacttactggttctgctgggttgtaaccggtatggcagacgtggtggcgatcacggcttatgctcagttctggttccccgatctctccgactgggtcgcctcgctggcggtgatagtgctgctgctgacgctcaatctcgccaccgtgaaaatgttcggtgagatggagttctggtttgcgatgatcaaaatcgtcgccattgtgtcgctgattgtcgtcggcctggtcatggtggcgatgcactttcagtcaccgactggtgtggaagcgtcattcgcgcatttgtggaatgacggcggctggttcccgaaaggtttaagtggcttctttgccggattccagatagcggttttcgctttcgtggggattgagctggtaggtacaacagctgcggaaaccaaagatccagagaaatcactgccacgcgcgattaactccattccgatccgtatcattatgttctacgtcttcgcgctgattgtgattatgtccgtgacgccgtggagttcggtagtcccggagaaaagcccgtttgttgaactgttcgtgttggtagggctgcctgctgccgcaagcgtgatcaactttgtggtgctgacctctgcggcgtcttccgctaacagcggcgtcttctctaccagccgtatgctgtttggtctggcgcaggaaggtgtggcaccgaaagcgttcgctaaactttctaagcgcgcagtacccgcgaaagggctgacgttctcgtgtatctgtctgctcggcggcgtggtgatgttgtatgtgaatcctagtgtgattggcgcgttcacgatgattacaaccgtttccgcgattctgtttatgttcgtctggacgattatcctttgctcgtaccttgtgtatcgcaaacagcgtcctcatctacatgagaagtcgatctacaagatgccgctcggcaagctgatgtgctgggtatgtatggcgttctttgtgttcgtggtcgtgttgctgacactggaagatgacactcgccaggcgctgctggttaccccgctgtggtttatcgcgctggggttgggctggctgtttattggtaagaagcgggctgctgaactgcggaaataa

SEQ ID NO: 22 - DNA, 32 bp, Artificial Sequence
  primer
ctgatctaccatgagtgtttcctttcgttggg

SEQ ID NO: 23 - DNA, 32 bp, Artificial Sequence
  primer
aaggaaacactcatggtagatcaggtaaaagt

SEQ ID NO: 24 - DNA, 40 bp, Artificial Sequence
  primer
gtcctccactcgagactagtaactgctggaagtgattaaa

SEQ ID NO: 25 - DNA, 1985 bp, Artificial Sequence
  pcj7-cycA(Eco)
agcgctgcgcctgaactagtagaaacatcccagcgctactaatagggagcgttgaccttccttccacggaccggtaatcggagtgcctaaaaccgcatgcggcttaggctccaagataggttctgcgcggccgggtaatgcatcttctttagcaacaagttgaggggtaggtgcaaataagaacgacatagaaatcgtctcctttctgtttttaatcaacatacaccaccacctaaaaattccccgaccagcaagttcacagtattcgggcacaatatcgttgccaaaatattgtttcggaatatcatgggatacgtacccaacgaaaggaaacactcatggtagatcaggtaaaagtcgttgccgatgatcaggctccggctgaacagtcgctacggcgcaatctcacaaaccgacatattcagcttattgccattggcggtgccattggtacggggttgtttatggggtctggcaaaacgattagccttgccgggccgtcgatcattttcgtttatatgatcattggttttatgctctttttcgtgatgcgggcaatgggggaattgctgctttcgaatctggaatacaaatcttttagtgacttcgcttccgatttactcgggccgtgggcaggatatttcaccggctggacttactggttctgctgggttgtaaccggtatggcagacgtggtggcgatcacggcttatgctcagttctggttccccgatctctccgactgggtcgcctcgctggcggtgatagtgctgctgctgacgctcaatctcgccaccgtgaaaatgttcggtgagatggagttctggtttgcgatgatcaaaatcgtcgccattgtgtcgctgattgtcgtcggcctggtcatggtggcgatgcactttcagtcaccgactggtgtggaagcgtcattcgcgcatttgtggaatgacggcggctggttcccgaaaggtttaagtggcttctttgccggattccagatagcggttttcgctttcgtggggattgagctggtaggtacaacagctgcggaaaccaaagatccagagaaatcactgccacgcgcgattaactccattccgatccgtatcattatgttctacgtcttcgcgctgattgtgattatgtccgtgacgccgtggagttcggtagtcccggagaaaagcccgtttgttgaactgttcgtgttggtagggctgcctgctgccgcaagcgtgatcaactttgtggtgctgacctctgcggcgtcttccgctaacagcggcgtcttctctaccagccgtatgctgtttggtctggcgcaggaaggtgtggcaccgaaagcgttcgctaaactttctaagcgcgcagtacccgcgaaagggctgacgttctcgtgtatctgtctgctcggcggcgtggtgatgttgtatgtgaatcctagtgtgattggcgcgttcacgatgattacaaccgtttccgcgattctgtttatgttcgtctggacgattatcctttgctcgtaccttgtgtatcgcaaacagcgtcctcatctacatgagaagtcgatctacaagatgccgctcggcaagctgatgtgctgggtatgtatggcgttctttgtgttcgtggtcgtgttgctgacactggaagatgacactcgccaggcgctgctggttaccccgctgtggtttatcgcgctggggttgggctggctgtttattggtaagaagcgggctgctgaactgcggaaataaccgcattatcatgctggatggcgcaatgccatccagcttttagatcactcacccgccagcgcgcgtgggaacagtacattgttttccagactgatgtgatccatcaggtcatcaatcagttcattaatgccgttatacatcgctttccaggtggtgcaggcttctggcggcggtgtgacgttattggtggtgtgtttaatcacttccagcagttactagtctcgagtggaggac

SEQ ID NO: 26 - protein, 952 aa, Corynebacterium ammoniagenes
MDFIARHLGPDATESKDMLARVGYDSVEALVTSAIPQSISITDALNMPQALSETDAQAKLRAYADKNVVLKSFYGQGYSDTITPAVIRRGLVEDAGWYTAYTPYQPEISQGRLESLLNFQTMVQDLTGLPIANASLLDEASAVAEAVGLMSRAVKKGRRVLLDARLHPQVLTVAAERARAIDLEVEIADLSNGVVGEDLVGAVVAYTGTEGDIFDPRAVIEEIHGRGGLVSVAADLLSLLLLEGPGSFGADIVIGSSQRFGVPLFFGGPHAAFMAVTDKLKRQMPGRLVGVSVDSEGRPAYRLALQTREQHIRRERATSNICTAQALLANVAAMYAVYHGPEGLKEIANHVHSLAASFAGAVTTQGLKITSSEFFDTVTVAGVDAASIKFSLEKAGYLVRTIGEDKVSVSFGESATQGDVTVLADAFGAAAVDNADFPLPEALTRTTEVLTHEIFNSIHSETQMMRYLRKLGDKDLALDRTMIPLGSCTMKLNPTAAMEPITWPEFANVHPYSPEYATQGWRELIEELEGWLAELTGYAKVSIQPNAGSQGELAGLLAIRRYHVANGDTNRDIVLIPASAHGTNAASATLANLRVVVVKTAEDGSIDLEDLDAKIAKHGQNMAGIMITYPSTHGVFDPEVREVCDKIHAAGGQVYIDGANMNALTGWAQPGKFGGDVSHLNLHKTFTIPHGGGGPGVGPIGVAEHLIPFLPTDAAADELDPANPTPVEQGVPITASQFGSAGVLPITWAYIAMTGGEGLTSATAHAILGANYLARELSDSFPILFTGNEGLVAHECILDLRALTDASGVTAADVAKRLIDFGFHAPTLAFPVAGTLMVEPTESEDIAELDRFIEAMRTIRAEIQEIIDGKIAYEDSVIRHAPYTAPSVSSDDWEYSFSREKAAWPVPSLRLNKYFPPVRRLDEAYGDRNLVCSCPPPEAFDFDADTDSTEEA

SEQ ID NO: 27 - protein, 367 aa, Corynebacterium ammoniagenes
MSELRQSPLHAEHEKLGASFTAFGPWNMPLKYGKELDEHHAVRNAVGMFDLSHMGEIWVNGPDAAAFLSYALISNMETVKNGKAKYSMIVAEDGGIIDDLISYRFSDTKFLVVPNAGNTDVVWEAFNQRIEGFDVELNNESLDVAMIALQGPNAAKVLVEQVAEESKEEVENLPYYAATMAKVADVDTIVARTGYTGEDGFELMIYNADATKLWQLFIDQDGVTPCGLASRDSLRLEAGMPLYGNELSRDITPVEAGMGVAFKKKTADFVGAEVLRQRLEEGPKQVIKALTSSERRAARTGAEIYAGEQLVGTVTSGQPSPTLGHPIALALVDTAANLEEGAEVEVDIRGKRYPFTVTKTPFYSREK

SEQ ID NO: 28 - protein, 129 aa, Corynebacterium ammoniagenes
MANLPAEFTYSEDHEWINAAQDAIVGKTVRIGITSVAADRLGEVVFAELPAVGDSVTAGETCGEVESTKSVSDLYSPVTGTVTAVNETVHDDYEIINNDPFGEGWLFEVEVEELGEVMTADEYAAENGI

SEQ ID NO: 29 - protein, 353 aa, Corynebacterium ammoniagenes
MLRIEKKNAESPIEQKPRWIRNQVRTGPGYEDMKKRVAGAGLHTVCQEAGCPNIHECWESREATFLIGGDRCTRRCDFCDIATGKPQALDTDEPRRVSENIQEMNLNYATITGVTRDDLPDEGAWLYAEVVRKIHEKNPHTGVENLTPDFSGKPDLLQEVFEARPEVFAHNLETVPRIFKRIRPAFRYERSLDVLQQAHDFGLITKSNLILGMGETEEEIQEALRDMRSVGTDIITITQYLRPGPRFHPIERWVRPEEFIAHSEYAKELGFTVMSGPLVRSSYRAGKLYTQAMKARGWELPENLKHLEETSDGATAQEASSLLKKYGPSEETPVTSRMAKTPVGADKFTASIR

SEQ ID NO: 30 - protein, 273 aa, Corynebacterium ammoniagenes
MTAPRDPFFPADRSIRASTAPVEVRRLGRMDYQEAWDYQAEVAAQRARDEVADTLLVVEHPAVYTAGKRTQPEDMPTNGLPVINVDRGGRITWHGEGQLVVYPIIKLAEPVDVVDYVRRLEEAVIHTVREMGVTTAGRIDGRSGVWVPSTTAAKDPAASHRDRKIAALGIRITRGVTMHGLALNCDNILDYYEHIIACGIDDADITTLALELGRDVTVDDAVEPLLIALDDALAGRMVVADHTFASAPDPIKLANEKARQARAQSSLTDHAGS

SEQ ID NO: 31 - DNA, 2859 bp, Corynebacterium ammoniagenes
atggatttcattgcccgccaccttgggccagatgccacagaatctaaggacatgctggcgcgtgtgggttatgacagcgtagaagcgcttgtcacctcagcaatcccccagtccattagcatcacggatgcgcttaatatgccgcaggcattgagtgagaccgacgcacaagccaagcttcgcgcttacgctgataaaaatgtcgtgctgaagtctttctacggccagggctactcagacaccatcacccctgctgttattcgccgcggtttggtagaagacgctggttggtacaccgcttataccccataccagccagaaatttcccagggtcgccttgagtcgctgctgaacttccagaccatggttcaagacctcaccggcttgcctattgcgaatgcttctttgctggatgaggcatcggcagttgctgaggccgtgggtttgatgtctcgtgcggtcaagaagggccgccgcgtactgctcgacgcccgtttgcacccacaggttctcaccgtggcggcggagcgtgcccgagcaattgacctcgaagttgagattgctgacttgagcaacggcgtggttggcgaagacctcgtcggcgcagtagttgcctacaccggtacggagggcgatatttttgacccacgtgctgttatcgaagaaatccatggccgcggcggacttgtttccgtcgcggctgacttgttgtccttgctgcttctggaaggcccaggctcgttcggtgcagacattgtcattggttcctcccaacgctttggtgtgccgctcttctttggtggcccacacgctgctttcatggcagtaactgacaagctaaagcgtcagatgccaggccgtttggtcggcgtatcggtcgattctgagggccgtcctgcttaccgcttggcgctgcagactcgtgaacagcacatccgccgtgaacgcgcgacgtcaaatatttgtaccgcgcaggcacttctggccaacgtggctgccatgtacgccgtctaccacggtccagaaggcttgaaggagattgctaaccacgtgcactccttggctgcttcctttgccggtgcagttactactcagggtctgaagattacttcctcggagttcttcgacaccgttaccgttgccggcgttgatgccgcatccattaagttcagcttggaaaaggccggatacctggtgcgcaccattggcgaggataaggtttctgtctccttcggtgagtccgcaacccaaggcgatgttactgtcttggcggacgcctttggtgccgctgcagtagataatgcagatttcccactgcctgaagcactcacccgcaccaccgaggtgctcacccacgaaatctttaactccattcactccgaaacccagatgatgcgttacctgcgcaagctcggtgataaggatctggctctagatcgcaccatgattcctttgggctcatgcaccatgaagctcaacccaaccgcagccatggaaccgatcacctggccagaattcgccaatgttcacccttactcccctgaatacgcaacccagggctggcgtgagctcattgaagagttggaaggctggttggctgagctgaccggctacgccaaggtttctatccaaccaaacgctggttcccagggcgagctagctggtcttttggctatccgccgctaccacgtcgcaaatggtgacaccaaccgcgatatcgtgttgattcctgcgtccgcgcacggcaccaacgctgcctccgcgaccctggcaaatctgcgcgttgttgtggttaagaccgccgaagacggctccatcgatctggaagatctcgatgcgaagatcgccaagcatggtcagaacatggccggaatcatgatcacctacccatccactcacggcgtctttgacccagaggttcgtgaagtctgcgacaagatccatgccgctggcggccaggtctacattgatggcgcaaacatgaatgctttgactggttgggctcagccgggcaagttcggtggcgatgtctcgcacttgaacctgcacaagactttcaccattccgcacggcggtggcggcccaggtgttggaccaattggtgtcgctgagcacctcattccattcctgccaacggatgctgcagctgatgagctggatcctgctaacccaaccccagtagaacagggcgttccaattactgcttcgcagtttggttccgctggtgttctgccgattacctgggcatacatcgcaatgaccggtggcgagggtctaacctccgctactgcacacgccatcttgggtgctaactaccttgcgcgcgaactctccgattccttcccaattctgttcaccggtaatgaaggtcttgttgcgcacgagtgcattttggatctgcgcgcgctaaccgatgcctcaggcgttactgcagcagacgttgccaagcgtttgatcgactttggcttccacgctcctaccctcgcattcccagtggctggcaccttgatggtggaacctactgagtctgaggatattgctgaactggatcgtttcattgaagcaatgcgcaccatccgtgcggagattcaggaaatcatcgatggcaagatcgcatatgaagattcggtcatccgccacgcaccttacaccgcaccgtcagtctcaagcgatgattgggagtactcctttagccgtgaaaaggccgcatggccagttccttcactgcgtttgaacaagtacttcccaccggtacgccgcctggatgaagcttacggcgaccgcaacctggtgtgctcctgcccaccgccagaggcattcgacttcgatgccgacaccgattccaccgaggaggcttaa

SEQ ID NO: 32 - DNA, 1104 bp, Corynebacterium ammoniagenes
atgtcagaactacgccagtccccactgcacgcagagcacgaaaagctcggcgcatcctttaccgcttttggcccttggaatatgccactaaagtacggcaaggagctcgatgagcaccacgcagtgcgtaatgcagtcggcatgtttgacctctcgcacatgggtgagatttgggtcaacggcccagacgccgctgcatttttgtcctatgcgctgatctccaacatggagaccgtgaaaaatggcaaggcgaagtactccatgattgttgctgaagacggcggcatcatcgatgacctaatttcctaccgtttctccgataccaagttcttggtagtgccaaacgctggcaacactgatgtggtttgggaagcttttaatcagcgcattgaaggcttcgatgtagaactcaacaatgagtccttggatgttgcgatgattgccctgcagggccccaatgctgccaaggttctagttgaacaggttgctgaagagtccaaggaagaagtagaaaaccttccttactatgccgcaaccatggccaaagtcgcagacgttgacaccatcgtcgcgcgcaccggctacaccggcgaagacggcttcgagctgatgatctacaacgccgatgcgaccaagctctggcagcttttcatcgaccaagatggtgttactccatgcggtttagcttcacgcgattccttgcgcttggaagctggcatgcctttgtacggcaatgagctttcccgcgatatcacccctgtcgaggcaggcatgggtgtggcgtttaagaagaagaccgctgacttcgtcggcgccgaggtcctgcgtcaacgcttggaagaaggccctaagcaagttatcaaggctttgacctcctctgagcgccgtgcagcgcgcaccggtgctgaaatctatgccggcgagcagttggtaggcaccgtaacttcgggtcagccatcgccgacgctgggacaccctattgccctggcactggtagatactgcagcaaacctcgaagaaggcgcagaagtagaagtggatattcgcggcaagcgttaccccttcaccgttaccaagacgcctttctatagccgcgagaagtaa

SEQ ID NO: 33 - DNA, 390 bp, Corynebacterium ammoniagenes
atggctaacctacctgcagaatttacttactccgaagaccacgagtggattaacgccgctcaggacgcaatcgttggcaaaactgttcgcatcggcatcacttctgttgccgcagaccgtcttggtgaggttgtcttcgctgagcttccagcagttggcgatagcgtcactgcaggtgaaacctgtggtgaggttgaatccaccaagtccgtttctgacctgtacagccctgtcaccggtaccgtgaccgctgtgaacgagacagtgcacgatgattatgaaatcatcaacaatgatcctttcggtgaaggttggctgtttgaggtcgaggttgaagaactcggcgaggttatgaccgctgatgaatacgcggcagaaaacggcatctaa

SEQ ID NO: 34 - DNA, 1062 bp, Corynebacterium ammoniagenes
atgctccgcattgaaaagaagaatgcggagtcacccattgagcagaagccgaggtggatccgcaaccaggtccgcactggccctggttatgaggacatgaaaaagcgtgttgctggcgctggcctgcacactgtctgccaggaggcaggctgtcctaatatccacgaatgctgggagtcccgagaagccaccttccttatcggcggtgaccgttgtactcgccgttgtgacttctgcgatattgccactggtaagccgcaggcattggacaccgatgagccgcgtcgcgtttcggaaaacatccaagagatgaatctgaactacgccacgatcactggtgttacccgtgatgaccttccagatgagggcgcatggctatatgctgaagtggttcgtaagatccacgagaagaaccctcacacgggtgtagaaaacctcaccccggacttctccggcaagccagacctgctgcaggaagtcttcgaggctcgccctgaggttttcgctcacaacttggaaaccgttcctcgtattttcaagcgtatccgcccggcattccgttatgagcgttctctggatgttttgcagcaggcacacgacttcggcctgatcaccaagtcgaacttgatcttgggcatgggtgaaactgaggaagagattcaggaagctctacgcgatatgcgctctgtgggcactgacatcattaccattacgcagtacctgcgtcctggtcctcgtttccacccaattgagcgttgggttcgccctgaggagttcattgcgcactccgagtacgccaaggaattgggctttaccgttatgtctggtcctttggttcgttcttcttaccgcgcgggcaagctctacacccaggcgatgaaggcacgtggctgggagctgccagaaaatctcaagcacctggaagaaacttctgatggcgcaaccgctcaggaagcttcgtcgctgttgaagaagtacggcccttccgaggaaacgccagttacttcccgcatggcaaagacgcctgtgggtgcagataaatttactgctagcatccgctaa

SEQ ID NO: 35 - DNA, 822 bp, Corynebacterium ammoniagenes
atgactgctccgcgtgacccgtttttccccgctgatcgttctattcgcgcttctactgccccggtagaggtgagacgtttaggtcgtatggattatcaagaagcctgggactatcaagcagaagtcgcagcgcagcgcgcacgtgacgaagttgcagacacgttgctggtcgttgagcaccccgctgtgtatacggcgggcaagcgcacgcagcccgaagatatgcccaccaacggtcttccggttatcaatgttgatcgtggcggccggattacctggcacggcgagggccagttggtggtctacccgattatcaagttggcagagcctgtcgatgtcgtcgattatgtccgccgtctggaagaagctgttattcataccgttcgggaaatgggggtaacaactgctgggcgtatcgatggtcgctcaggcgtgtgggtgccatcgactaccgctgcgaaagacccggcagcatcgcaccgagaccgcaagattgcggccttaggcatccgcattacgcgtggggttaccatgcatggtctggcgctcaattgcgacaatatcttggactactacgagcacattattgcctgcggtattgatgatgccgatatcaccaccctggcgctagagctgggccgcgacgtcaccgtagatgatgcggttgagcccttgctcattgcgcttgacgatgccttggccggccgcatggtcgtcgccgaccacactttcgcatctgccccagaccccatcaaattagctaatgagaaagcgcgccaagcacgcgcgcagtcttccttgactgatcatgcaggctcttaa

SEQ ID NO: 36 - DNA, 40 bp, Artificial Sequence
  primer
gtacccggggatcctctagacaaagccgaagagaagttgg

SEQ ID NO: 37 - DNA, 32 bp, Artificial Sequence
  primer
gcatagagtactcggcgcccataaatttcact

SEQ ID NO: 38 - DNA, 32 bp, Artificial Sequence
  primer
gcgccgagtactctatgccgagaagttgaaca

SEQ ID NO: 39 - DNA, 40 bp, Artificial Sequence
  primer
gcctgcaggtcgactctagactgggcatcacactattttt

SEQ ID NO: 40 - DNA, 531 bp, Artificial Sequence
  del-N2131L
gtacccggggatcctctagacaaagccgaagagaagttggcttgacggacccgaaattccagctgattttgacgatcctgatgcacccggcaggtggcctggcgaaaagttggggcttcctcaagaaggggccggctctctgtcctcagtggctcgtcgtatcggcggggtctgcgtggactggggtgtttcctgggttattgctattgtgctgtccaatttcacggatgtgctgggcgatgtagcgacatccacgctcattattttcgtgatcctgggttggcttaccggttggatctttgctcgcaccccaggtcatgccgtgtttggcatgggccttgcgcgtgtggatgcagaggaacgtgtgggctggtggcgtgcgctggttcgcccactgctgacgatcttgattctgcctgccgtgatggtggatgctgacggccgtgggctccacgacaaggcaacgggaactgcagttatccgcgggtaatttgtcttgagtgaaatttatgggcgccgagtactctatgc

SEQ ID NO: 41 - DNA, 555 bp, Artificial Sequence
  del-N2131R
gcgccgagtactctatgccgagaagttgaacagcaatcttgcagcaactcctcagtattcacaccagccccaatggacacaaaaacatcagccccagaatcgcccctaagggcctcaaatacacgatctggaccaactagtcatcgggaaaacccaaccccttaaatcgccttctgcgcttaaggggtcaatgctagataagtaggaacaacaacgtttgggcggccaggatctttgcgatcatcgccagcggatacacagaggtataacccatggcagggagctcgttgcgggaggcatctgacacataactcagcacagcagggtgggtttgcgtaccggcgaggatgccagcggtttcaccgaaggggattttcatcagtttgtggccaacgaacagcaccgtgatggagatgaacaaagtgagcagcgcaccgaagccgatgatggtgagtgattgggggtcgctgatcgctgatcgaaatcctgcgcccgctgaggtaccgatggcagccaaaaatagtgtgatgcccagtctagagtcgacctgcaggc

SEQ ID NO: 42 - DNA, 40 bp, Artificial Sequence
  primer
gaaatttatgggcgccgagtcaggatgcaattgccatccg

SEQ ID NO: 43 - DNA, 32 bp, Artificial Sequence
  primer
gccattagcgtgttacttctcgcggctataga

SEQ ID NO: 44 - DNA, 4499 bp, Artificial Sequence
  Pn_gcvPT(Cam)
gaaatttatgggcgccgagtcaggatgcaattgccatccgtcagatgtgctacttgccatttacctacgaccaccaggttgtagatggtgcagatgcaggtcgcttcatcaccaccatcaaggaccgccttgaaaccggtaacttcgaatctgacttggctctctaagtccagtttcagaatgtacggtagcttctagccgtttgagttagaagcaaaagccaccatgtagaaatttctacatggtggctttgtggttttctcatctctaagttctcattttcgggggtataaaaaactaacaaatccgcattgcagtcttcacattttcttcaaatctggcgttcatgctggttataacagcgctttcgcaggagccattacaaccggcggctaggcgtgggtagaaaagcaatgaaaagccggggctagccatataatgaggaatgttgactgccttcgtgcggtcgctttagcaagcctggacaaatgtatcgttcctccaaaggagtttgacacttatggatttcattgcccgccaccttgggccagatgccacagaatctaaggacatgctggcgcgtgtgggttatgacagcgtagaagcgcttgtcacctcagcaatcccccagtccattagcatcacggatgcgcttaatatgccgcaggcattgagtgagaccgacgcacaagccaagcttcgcgcttacgctgataaaaatgtcgtgctgaagtctttctacggccagggctactcagacaccatcacccctgctgttattcgccgcggtttggtagaagacgctggttggtacaccgcttataccccataccagccagaaatttcccagggtcgccttgagtcgctgctgaacttccagaccatggttcaagacctcaccggcttgcctattgcgaatgcttctttgctggatgaggcatcggcagttgctgaggccgtgggtttgatgtctcgtgcggtcaagaagggccgccgcgtactgctcgacgcccgtttgcacccacaggttctcaccgtggcggcggagcgtgcccgagcaattgacctcgaagttgagattgctgacttgagcaacggcgtggttggcgaagacctcgtcggcgcagtagttgcctacaccggtacggagggcgatatttttgacccacgtgctgttatcgaagaaatccatggccgcggcggacttgtttccgtcgcggctgacttgttgtccttgctgcttctggaaggcccaggctcgttcggtgcagacattgtcattggttcctcccaacgctttggtgtgccgctcttctttggtggcccacacgctgctttcatggcagtaactgacaagctaaagcgtcagatgccaggccgtttggtcggcgtatcggtcgattctgagggccgtcctgcttaccgcttggcgctgcagactcgtgaacagcacatccgccgtgaacgcgcgacgtcaaatatttgtaccgcgcaggcacttctggccaacgtggctgccatgtacgccgtctaccacggtccagaaggcttgaaggagattgctaaccacgtgcactccttggctgcttcctttgccggtgcagttactactcagggtctgaagattacttcctcggagttcttcgacaccgttaccgttgccggcgttgatgccgcatccattaagttcagcttggaaaaggccggatacctggtgcgcaccattggcgaggataaggtttctgtctccttcggtgagtccgcaacccaaggcgatgttactgtcttggcggacgcctttggtgccgctgcagtagataatgcagatttcccactgcctgaagcactcacccgcaccaccgaggtgctcacccacgaaatctttaactccattcactccgaaacccagatgatgcgttacctgcgcaagctcggtgataaggatctggctctagatcgcaccatgattcctttgggctcatgcaccatgaagctcaacccaaccgcagccatggaaccgatcacctggccagaattcgccaatgttcacccttactcccctgaatacgcaacccagggctggcgtgagctcattgaagagttggaaggctggttggctgagctgaccggctacgccaaggtttctatccaaccaaacgctggttcccagggcgagctagctggtcttttggctatccgccgctaccacgtcgcaaatggtgacaccaaccgcgatatcgtgttgattcctgcgtccgcgcacggcaccaacgctgcctccgcgaccctggcaaatctgcgcgttgttgtggttaagaccgccgaagacggctccatcgatctggaagatctcgatgcgaagatcgccaagcatggtcagaacatggccggaatcatgatcacctacccatccactcacggcgtctttgacccagaggttcgtgaagtctgcgacaagatccatgccgctggcggccaggtctacattgatggcgcaaacatgaatgctttgactggttgggctcagccgggcaagttcggtggcgatgtctcgcacttgaacctgcacaagactttcaccattccgcacggcggtggcggcccaggtgttggaccaattggtgtcgctgagcacctcattccattcctgccaacggatgctgcagctgatgagctggatcctgctaacccaaccccagtagaacagggcgttccaattactgcttcgcagtttggttccgctggtgttctgccgattacctgggcatacatcgcaatgaccggtggcgagggtctaacctccgctactgcacacgccatcttgggtgctaactaccttgcgcgcgaactctccgattccttcccaattctgttcaccggtaatgaaggtcttgttgcgcacgagtgcattttggatctgcgcgcgctaaccgatgcctcaggcgttactgcagcagacgttgccaagcgtttgatcgactttggcttccacgctcctaccctcgcattcccagtggctggcaccttgatggtggaacctactgagtctgaggatattgctgaactggatcgtttcattgaagcaatgcgcaccatccgtgcggagattcaggaaatcatcgatggcaagatcgcatatgaagattcggtcatccgccacgcaccttacaccgcaccgtcagtctcaagcgatgattgggagtactcctttagccgtgaaaaggccgcatggccagttccttcactgcgtttgaacaagtacttcccaccggtacgccgcctggatgaagcttacggcgaccgcaacctggtgtgctcctgcccaccgccagaggcattcgacttcgatgccgacaccgattccaccgaggaggcttaaatcaatgtcagaactacgccagtccccactgcacgcagagcacgaaaagctcggcgcatcctttaccgcttttggcccttggaatatgccactaaagtacggcaaggagctcgatgagcaccacgcagtgcgtaatgcagtcggcatgtttgacctctcgcacatgggtgagatttgggtcaacggcccagacgccgctgcatttttgtcctatgcgctgatctccaacatggagaccgtgaaaaatggcaaggcgaagtactccatgattgttgctgaagacggcggcatcatcgatgacctaatttcctaccgtttctccgataccaagttcttggtagtgccaaacgctggcaacactgatgtggtttgggaagcttttaatcagcgcattgaaggcttcgatgtagaactcaacaatgagtccttggatgttgcgatgattgccctgcagggccccaatgctgccaaggttctagttgaacaggttgctgaagagtccaaggaagaagtagaaaaccttccttactatgccgcaaccatggccaaagtcgcagacgttgacaccatcgtcgcgcgcaccggctacaccggcgaagacggcttcgagctgatgatctacaacgccgatgcgaccaagctctggcagcttttcatcgaccaagatggtgttactccatgcggtttagcttcacgcgattccttgcgcttggaagctggcatgcctttgtacggcaatgagctttcccgcgatatcacccctgtcgaggcaggcatgggtgtggcgtttaagaagaagaccgctgacttcgtcggcgccgaggtcctgcgtcaacgcttggaagaaggccctaagcaagttatcaaggctttgacctcctctgagcgccgtgcagcgcgcaccggtgctgaaatctatgccggcgagcagttggtaggcaccgtaacttcgggtcagccatcgccgacgctgggacaccctattgccctggcactggtagatactgcagcaaacctcgaagaaggcgcagaagtagaagtggatattcgcggcaagcgttaccccttcaccgttaccaagacgcctttctatagccgcgagaagtaacacgctaatggc

SEQ ID NO: 45 - DNA, 32 bp, Artificial Sequence
  primer
cgcgagaagtaacacgctaatggcgcattgaa

SEQ ID NO: 46 - DNA, 40 bp, Artificial Sequence
  primer
tcaacttctcggcatagagtttagcggatgctagcagtaa

SEQ ID NO: 47 - DNA, 3053 bp, Artificial Sequence
  Pn_gcvH-lipBA(Cam)
cgcgagaagtaacacgctaatggcgcattgaatcgccggccgatatttcagggcttgtacgaaaacggcatcatcgaagcagatggcacccaagttgaagtcgacactatcatttgggcgattggctttcgtcccagcctggcgcacttggacccgctgaacctttataacgagaagggcgggatcaacattgagggcacaaaggtaaagggaaagaagaatcttcacctaattggctatggcccttcccaatccacagtaggagctaaccgggcaggtcgagctgccgtcatcagcattgcccgcgacctgaaatctggcacctaggcactgctcaatgtccgatgggagcaaactcttaacccccgaaggggcaatatcaacgtgcgatagtggcatgtaaagtgcttcgctgcgctaaagtgaaagcgatacttcaacccatgcacctattcgctgcattcccggcggatcaggcacgtcaatgaaaggatagttcctatggctaacctacctgcagaatttacttactccgaagaccacgagtggattaacgccgctcaggacgcaatcgttggcaaaactgttcgcatcggcatcacttctgttgccgcagaccgtcttggtgaggttgtcttcgctgagcttccagcagttggcgatagcgtcactgcaggtgaaacctgtggtgaggttgaatccaccaagtccgtttctgacctgtacagccctgtcaccggtaccgtgaccgctgtgaacgagacagtgcacgatgattatgaaatcatcaacaatgatcctttcggtgaaggttggctgtttgaggtcgaggttgaagaactcggcgaggttatgaccgctgatgaatacgcggcagaaaacggcatctaagccttttttacttttcacacctttaagcgcggagcagtttgccccaagtactacactgggaaaacatgactgctccgcgtgacccgtttttccccgctgatcgttctattcgcgcttctactgccccggtagaggtgagacgtttaggtcgtatggattatcaagaagcctgggactatcaagcagaagtcgcagcgcagcgcgcacgtgacgaagttgcagacacgttgctggtcgttgagcaccccgctgtgtatacggcgggcaagcgcacgcagcccgaagatatgcccaccaacggtcttccggttatcaatgttgatcgtggcggccggattacctggcacggcgagggccagttggtggtctacccgattatcaagttggcagagcctgtcgatgtcgtcgattatgtccgccgtctggaagaagctgttattcataccgttcgggaaatgggggtaacaactgctgggcgtatcgatggtcgctcaggcgtgtgggtgccatcgactaccgctgcgaaagacccggcagcatcgcaccgagaccgcaagattgcggccttaggcatccgcattacgcgtggggttaccatgcatggtctggcgctcaattgcgacaatatcttggactactacgagcacattattgcctgcggtattgatgatgccgatatcaccaccctggcgctagagctgggccgcgacgtcaccgtagatgatgcggttgagcccttgctcattgcgcttgacgatgccttggccggccgcatggtcgtcgccgaccacactttcgcatctgccccagaccccatcaaattagctaatgagaaagcgcgccaagcacgcgcgcagtcttccttgactgatcatgcaggctcttaagtagtgccccctattcgggcagcgatgtccattcttggaatatttcaggaaccggtgggattgaaggaattaaggccttaagcgcacccatcaacggggtcaaaaatacgaggtcagtatcataattagttgaatttaaaatcacacaacacgtagggtggaatttgtgactattaagcctgaaggacgcaagatgctccgcattgaaaagaagaatgcggagtcacccattgagcagaagccgaggtggatccgcaaccaggtccgcactggccctggttatgaggacatgaaaaagcgtgttgctggcgctggcctgcacactgtctgccaggaggcaggctgtcctaatatccacgaatgctgggagtcccgagaagccaccttccttatcggcggtgaccgttgtactcgccgttgtgacttctgcgatattgccactggtaagccgcaggcattggacaccgatgagccgcgtcgcgtttcggaaaacatccaagagatgaatctgaactacgccacgatcactggtgttacccgtgatgaccttccagatgagggcgcatggctatatgctgaagtggttcgtaagatccacgagaagaaccctcacacgggtgtagaaaacctcaccccggacttctccggcaagccagacctgctgcaggaagtcttcgaggctcgccctgaggttttcgctcacaacttggaaaccgttcctcgtattttcaagcgtatccgcccggcattccgttatgagcgttctctggatgttttgcagcaggcacacgacttcggcctgatcaccaagtcgaacttgatcttgggcatgggtgaaactgaggaagagattcaggaagctctacgcgatatgcgctctgtgggcactgacatcattaccattacgcagtacctgcgtcctggtcctcgtttccacccaattgagcgttgggttcgccctgaggagttcattgcgcactccgagtacgccaaggaattgggctttaccgttatgtctggtcctttggttcgttcttcttaccgcgcgggcaagctctacacccaggcgatgaaggcacgtggctgggagctgccagaaaatctcaagcacctggaagaaacttctgatggcgcaaccgctcaggaagcttcgtcgctgttgaagaagtacggcccttccgaggaaacgccagttacttcccgcatggcaaagacgcctgtgggtgcagataaatttactgctagcatccgctaaactctatgccgagaagttga

SEQ ID NO: 48 - protein, 281 aa, Artificial Sequence
  C.gl HisG variant
MLKIAVPNKGSLSERAMEILAEAGYAGRGDSKSLNVFDEANNVEFFFLRPKDIAIYVAGGQLDLGITGRDLARDSQADVHEVLSLGFGSSTFRYAAPADEEWSIEKLDGKRIATSYPNLVRDDLAARGLSAEVLRLDGAVEVSIKLGVADAIADVVSTGRTLRQQGLAPFGEVLCTSEAVIVGRKDEKVTPEQQILLRRIQGILHAQNFLMLDYNVDRDNLDAATAVTPGLSHPQVSPLARDNWVAVRAMVPRRSANAIMDKLAGLGAEAILASEIRIARI

SEQ ID NO: 49 - DNA, 35 bp, Artificial Sequence
  primer
tcgagctcggtacccatcgccatctacgttgctgg

SEQ ID NO: 50 - DNA, 35 bp, Artificial Sequence; identical to SEQ ID NO: 49
  primer

SEQ ID NO: 51 - DNA, 45 bp, Artificial Sequence
  primer
gtgccagtggggatacctgtgggtgggataagcctggggttactg

SEQ ID NO: 52 - DNA, 45 bp, Artificial Sequence
  primer
aaccccaggcttatcccacccacaggtatccccactggcacgcga

SEQ ID NO: 53 - DNA, 35 bp, Artificial Sequence
  primer
ctctagaggatccccgggacgtggttgatggtggt

SEQ ID NO: 54 - DNA, 35 bp, Artificial Sequence; identical to SEQ ID NO: 49
  primer

SEQ ID NO: 55 - DNA, 35 bp, Artificial Sequence
  primer
gagtctagaagtactcgagatgctgacctcgtttc

SEQ ID NO: 56 - DNA, 35 bp, Artificial Sequence
  primer
agcatctcgagtacttctagactcgcacgaaaaag

SEQ ID NO: 57 - DNA, 35 bp, Artificial Sequence
  primer
ctctagaggatcccctttgggcagagctcaaattc

SEQ ID NO: 58 - DNA, 40 bp, Artificial Sequence
  primer
gcatgggcttgctgctcgagttcagggtagttgactaaag

SEQ ID NO: 59 - DNA, 20 bp, Artificial Sequence
  primer
agtttcgtaacccaccttgc

SEQ ID NO: 60 - DNA, 20 bp, Artificial Sequence
  primer
cgcttctcaatctgatgaga

SEQ ID NO: 61 - DNA, 35 bp, Artificial Sequence
  primer
gtcagcatctcgagtgctccttagggagccatctt

SEQ ID NO: 62 - DNA, 35 bp, Artificial Sequence
  primer
gtcaaatgtcttcacatgtgtgcacctttcgatct

SEQ ID NO: 63 - DNA, 35 bp, Artificial Sequence
  primer
gaaaggtgcacacatgtgaagacatttgactcgct

SEQ ID NO: 64 - DNA, 35 bp, Artificial Sequence
  primer
tcgtttttaggcctcctagatgcgggcgatgcgga

SEQ ID NO: 65 - DNA, 35 bp, Artificial Sequence
  primer
atcgcccgcatctaggaggcctaaaaacgaccgag

SEQ ID NO: 66 - DNA, 35 bp, Artificial Sequence
  primer
gacagttttggtcatgttgtgtctcctctaaagat

SEQ ID NO: 67 - DNA, 35 bp, Artificial Sequence
  primer
tagaggagacacaacatgaccaaaactgtcgccct

SEQ ID NO: 68 - DNA, 35 bp, Artificial Sequence
  primer
tgaagcgccggtaccgcttacagcaaaacgtcatt

SEQ ID NO: 69 - DNA, 35 bp, Artificial Sequence
  primer
cgttttgctgtaagcggtaccggcgcttcatgtca

SEQ ID NO: 70 - DNA, 35 bp, Artificial Sequence
  primer
agtgacattcaacattgttttgatctcctccaata

SEQ ID NO: 71 - DNA, 35 bp, Artificial Sequence
  primer
gaggagatcaaaacaatgttgaatgtcactgacct

SEQ ID NO: 72 - DNA, 35 bp, Artificial Sequence
  primer
cgctgggatgtttctctagagcgctcccttagtgg

SEQ ID NO: 73 - DNA, 35 bp, Artificial Sequence
  primer
aagggagcgctctagagaaacatcccagcgctact

SEQ ID NO: 74 - DNA, 35 bp, Artificial Sequence
  primer
agtcatgccttccatgagtgtttcctttcgttggg

SEQ ID NO: 75 - DNA, 35 bp, Artificial Sequence
  primer
cgaaaggaaacactcatggaaggcatgactaatcc

SEQ ID NO: 76 - DNA, 35 bp, Artificial Sequence
  primer
cgagtctagaagtgcctattttaaacgatccagcg